(19) United States
(12) Patent Application Publication
(10) Pub. No.: US 2023/0301646 A1
(43) Pub. Date: Sep. 28, 2023

(54) COMPRESSION SYSTEMS AND METHODS FOR FRACTURES AND FUSIONS

(71) Applicants: Naven DUGGAL, Manlius, NY (US); Dinesh KOKA, Winter Park, FL (US); Douglas LEACH, Cresskill, NJ (US)

(72) Inventors: Naven DUGGAL, Manlius, NY (US); Dinesh KOKA, Winter Park, FL (US); Douglas LEACH, Cresskill, NJ (US)

(21) Appl. No.: 18/125,652

(22) Filed: Mar. 23, 2023

Related U.S. Application Data
(60) Provisional application No. 63/323,016, filed on Mar. 23, 2022.
(60) Provisional application No. 63/323,404, filed on Mar. 24, 2022.

(51) Int. Cl. A61B 17/04

(52) U.S. Cl. CPC A61B 17/0401; A61B 2017/0409; A61B 2017/0445

(57) ABSTRACT

A systems may compress a first bone portion relative to a second bone portion. The system may have an anchor moveable through a hole in the first bone portion or the second bone portion. The anchor includes a proximal component that has a proximal component proximal end with a head, and a proximal component distal end with a bore. The anchor also includes a distal component that has a distal component proximal end, including a shaft that may be translatably retained within the bore. The distal component also has a distal component distal end with a toggle mechanism that is moveable and/or actuatable. The toggle mechanism may move between a retracted position, in which the toggle mechanism may be inserted through the hole in the bone, and a deployed position, in which the toggle mechanism may extend beyond the hole, thereby restricting motion and applying compressive force to the bone.

### CROSS-REFERENCE TO RELATED APPLICATIONS

This application claims the benefit of U.S. Provisional Patent Application Ser. No. 63/323,016, entitled “FRACTURE/FUSION COMPRESSION SYSTEMS AND METHODS,” filed on Mar. 23, 2022, and U.S. Provisional Patent Application Ser. No. 63/323,404, entitled “FRACTURE/FUSION COMPRESSION SYSTEMS AND METHODS,” filed on Mar. 24, 2022, the disclosures of which are incorporated herein by reference in their entirety.

This application is related to U.S. patent application Ser. No. 12/835,050 filed on Jul. 13, 2010, entitled “JOINT ARTHRODESIUS AND ARTHROPLASTY,” which issued on Aug. 19, 2014 as U.S. Pat. No. 8,808,336. This application is related to U.S. patent application Ser. No. 12/835,017 filed on Jul. 13, 2010, entitled “JOINT ARTHRODESIS AND ARTHROPLASTY,” which issued on Apr. 21, 2015 as U.S. Pat. No. 9,011,503. This application is related to U.S. patent application Ser. No. 13/964,945 filed on Aug. 12, 2013, entitled “JOINT ARTHRODESIS AND ARTHROPLASTY,” which issued on Jul. 12, 2016 as U.S. Pat. No. 9,387,019. This application is related to U.S. patent application Ser. No. 15/170,810 filed on Jun. 1, 2016, entitled “JOINT ARTHRODESIUS AND ARTHROPLASTY,” which issued on May 8, 2018 as U.S. Pat. No. 9,962,201. The foregoing are incorporated by reference as though as set forth herein in their entirety.

### TECHNICAL FIELD

The present disclosure relates to surgical implants for facilitating fusion of bones and/or healing of bone fractures. More specifically, the present disclosure relates to implants maintaining compression across two bones or bone fragments.

### BACKGROUND

Arthritis, and similar maladies, such as pain, inflammation, and swelling, may be the result of smooth cartilage wearing away in a joint of a patient. These symptoms are common following traumatic injuries, such as a fracture. Different treatments and/or implants may be recommended based on the affected joint. For example, two bones of the foot—the talus and the calcaneus—meet at the subtalar joint. The tibia (i.e., shinbone) rests on the top of the talus. Together, these bones form the tibiotalar joint. A surgeon may prescribe ankle fusion to stop the pain and swelling and provide stability to the joint after a patient has experienced an ankle fracture.

Fusion, or arthrodesis, often includes the use of plates, screws, nails, and other hardware to compress the bones together. An arthrodesis procedure may be the recommended procedure for patients with Diabetic Charcot Arthropathy, for post traumatic patients with poor bone stock, for young patients experiencing arthritis, or for patients who have experienced a failed ankle arthroplasty (e.g., failed replacement of distal tibia or a portion of the talus). However, there are disadvantages associated with the devices, systems, and methods for current arthrodesis, joint fusion, stabilization, and/or fixation.

Unfortunately, current systems that achieve ankle fusion, stabilization, and/or fixation may use cannulated screws that are prone to hardware failure or lack adequate compression across the fusion site. By way of another example, the screws may be limited in the motion that they are able to restrict, meaning they may not restrict motion in the plane of motion of the joint. This inadequacy may increase the likelihood of development of nonunion, failure of the arthrodesis, or failure to fully address/alleviate the initial malady. In yet another example, some plate systems are often able to accommodate deformity only in one plane, or they may cause prominence that leads to postoperative skin irritation.

Further, many existing systems rely on bone screws that can strip the bone, particularly when seated in poor quality bone. Yet further, another detriment to existing procedures, such as the use of retrograde nails in the scenario of ankle joint arthritis, is that they may require immobilization and/or fusion of the uninvolved joint (for example, the subtalar joint), which leads to unnecessary restriction of foot and ankle motion.

Furthermore, current retrograde systems do not adequately stabilize the posterior facet joint because they do not fuse that area with the fusion nail. These systems, and similar systems and procedures, may also require an eight- to twelve-week period of restrictions, including elimination of weight bearing activities, which may be difficult to achieve for patients.

With current disadvantages and poor patient outcomes, current ankle arthroplasty may not be an option for patients with poor bone stock. This would include those who may be undergoing repeat surgery and diabetic patients with Charcot arthropathy. For example, procedures requiring large bone cuts may not be an option for these types of patients as they generally create large voids which are difficult or impossible to fill if the ankle replacement fails. Furthermore, fusion procedures following an arthroplasty with large bone cuts are often associated with increased rates of nonunion.

Therefore, despite the improvements and advantages obtained by current systems and methods, the need still exists for improved ankle arthrodesis systems that provide improved compression, structural bone support, long-term fusion, fracture fixation stability, and/or pain relief. Furthermore, it would be even more advantageous if these improvements and advantages were not restricted to the ankle or foot of a patient. The need further exists for systems and methods for maintaining compression across any joint to be fused, or across fragments of a bone to be rejoined, in order to facilitate the healing and/or fusion process. Midfoot and hindfoot arthrodesis are additional examples.

### SUMMARY

The various systems and methods of the present disclosure have been developed in response to the present state of the art, and in particular, in response to the problems and needs in the art that have not yet been fully solved by currently available surgical instruments, devices, systems, and methods for implanting bone anchor assemblies in a patient.

According to some embodiments, a system for providing compressive force relative to a first bone portion and a second bone portion may have an anchor insertable into a hole formed through at least part of the first bone portion and the second bone portion. The anchor may have a proximal component with a proximal component proximal end with a head, and a proximal component distal end with a proximal coupling feature. The anchor may further include a distal component with a distal component proximal end with a distal coupling feature that is configured to be translatably coupled to the proximal coupling feature, and a distal component distal end comprising a toggle mechanism that is movable between a retracted position, in which the toggle mechanism is insertable through the hole, and a deployed position, in which the toggle mechanism extends beyond a diameter of the hole to restrict motion of the toggle mechanism through the hole. The anchor may further have an actuator configured to be operatively coupled to the toggle mechanism such that the actuator can be actuated by a user to trigger motion of the toggle mechanism between the retracted position and the deployed position.

The system may further have an inserter with a grip member and a biasing member configured to compress to transmit rotation of the grip member to the anchor.

The toggle mechanism may have a curved elongated member.

The curved elongated member may be formed of a superelastic material.

The toggle mechanism may have an elongated member. The actuator may have a suture coupled to the elongated member such that tension on the suture causes the elongated member to rotate from the retracted position to the deployed position.

The proximal coupling feature may have internal threads, and the distal coupling feature may have a male member having proximal external threads configured to translatably engage the internal threads of the proximal coupling feature.

According to some embodiments, a system may provide compressive force relative to a first bone portion and a second bone portion. The system may have an anchor insertable into a hole formed through at least part of the first bone portion and the second bone portion. The anchor may have a proximal component with a proximal component proximal end having a head, and a proximal component distal end having a proximal coupling feature. The anchor may further have a distal component with a distal component proximal end with a distal coupling feature that is configured to be translatably coupled to the proximal coupling feature, and a distal component distal end with a toggle mechanism that is actuatable to move between a retracted position, in which the toggle mechanism is insertable through the hole, and a deployed position, in which the toggle mechanism extends beyond a diameter of the hole to restrict motion of the toggle mechanism through the hole. The anchor may be configured to elastically deform in response to exertion of the compressive force such that the anchor continues to exert at least part of the compressive force if the first bone portion and the second bone portion move closer together after implantation of the anchor.

The toggle mechanism may have an elongated member that is pivotably coupled to a remainder of the distal component such that the elongated member is pivotable from the retracted position to the deployed position.

The elongated member may be configured to elastically deform in response to exertion of the compressive force.

The elongated member may have a central portion that is pivotably coupled to the remainder of the distal component, and two end portions that, in the deployed position, contact the second bone portion. The elongated member may be configured to elastically deform such that the end portions flex distally relative to the central portion.

Each of the end portions may have a spike that, in the deployed position, is oriented toward the second bone portion.

The elongated member may be formed of a superelastic material.

The proximal component distal end may have an internally-threaded bore. The distal component distal end may have a threaded post configured to threadably engage the internally-threaded bore.

According to some embodiments, system for providing compressive force relative to a first bone portion and a second bone portion may have an anchor moveable through a hole formed in at least part of the first bone portion or the second bone portion. The anchor may have a proximal component with a proximal component proximal end comprising a head, and a proximal component distal end with a bore. The anchor may further have a distal component with a distal component proximal end with a shaft that is configured to be translatably retained within the bore, and a distal component distal end with a toggle mechanism that is moveable between a retracted position, in which the toggle mechanism is insertable through the hole, and a deployed position, in which the toggle mechanism extends beyond a diameter of the hole to restrict motion of the toggle mechanism through the hole.

The first toggle mechanism may be elastically deformable to help maintain compression between the first bone portion and the second bone portion after implantation.

The system may further have a curved washer configured to encircle the proximal component and abut the first bone portion. The curved washer may be elastically deformable to further help maintain compression between the first bone portion and the second bone portion.

The curved washer may be a Nitinol washer.

The proximal component may have a maximum width, excluding the head, that is greater than a maximum width of the distal component such that the distal component fits within a distal portion of the hole with a distal diameter smaller than a proximal diameter of a proximal portion of the hole.

The system may further have an inserter configured to be coupled to the proximal component and to, with the anchor in the hole, actuate the distal component proximally to draw the second bone portion toward the first bone portion.

The system may further have an actuator configured to be operatively coupled to the toggle mechanism such that the actuator can be actuated by a user to trigger motion of the toggle mechanism between the retracted position and the deployed position.

These and other features and advantages of the present disclosure will become more fully apparent from the following description and appended claims, or may be learned by the practice of the systems and methods set forth hereinafter.

### BRIEF DESCRIPTION OF THE DRAWINGS

Exemplary embodiments of the disclosure will become more fully apparent from the following description and appended claims, taken in conjunction with the accompanying drawings. Understanding that these drawings depict only exemplary embodiments and are, therefore, not to be considered limiting of the scope of the appended claims, the exemplary embodiments of the present disclosure will be described with additional specificity and detail through use of the accompanying drawings in which:

FIG. 1A is a perspective view of a system for providing compressive force relative to a first bone portion and a second bone portion, according to an embodiment of the present disclosure;

FIG. 1B is a side view of the system of FIG. 1A;

FIG. 1C is a top view, from tip to handle, of the system of FIG. 1A;

FIG. 1D is a bottom view, from handle to tip, of the system of FIG. 1A;

FIG. 2 is an exploded view of an inserter assembly, according to an embodiment of the present disclosure;

FIG. 3 is an alternative exploded view of the inserter assembly of FIG. 2;

FIG. 4A is a perspective view of an anchor, according to an embodiment of the present disclosure;

FIG. 4B is another perspective view of the anchor of FIG. 4A;

FIG. 4C is a perspective view of a distal component of the anchor of FIG. 4A;

FIG. 5 is an exploded view of a moveable portion of the toggle mechanism, according to an embodiment of the present disclosure;

FIG. 6 is an alternative perspective view of the distal component of the anchor, according to an embodiment of the present disclosure;

FIG. 7 is a perspective view of a proximal component of the anchor of FIG. 4A;

FIG. 8 is a perspective view of a threaded component of the inserter assembly of FIG. 2;

FIG. 9 is a perspective view of a distal component of the inserter assembly of FIG. 2;

FIG. 10 is a perspective view of a biasing member of the inserter assembly of FIG. 2;

FIG. 11 is a perspective view of a hollow, cannulated grip member of the inserter assembly of FIG. 2;

FIG. 12 is a perspective view of a hollow, cannulated shaft member of the inserter assembly of FIG. 2;

FIG. 13 is a perspective view of another hollow, cannulated grip member of the inserter assembly of FIG. 3;

FIG. 14 is a perspective view of a pin member of the inserter assembly of FIG. 3;

FIG. 15 is a perspective view of another hollow, cannulated grip member of the inserter assembly of FIG. 3;

FIG. 16 is a perspective view of an anchor member, according to another embodiment of the present disclosure;

FIG. 17 is a perspective view of an anchor member, according to another embodiment of the present disclosure;

FIG. 18 is a perspective view of a retracted position of a toggle mechanism, according to an embodiment of the present disclosure;

FIG. 19 is a perspective view of a deployed position of a toggle mechanism, according to an embodiment of the present disclosure;

FIG. 20 is a perspective view of the anchor and an actuator, according to an embodiment of the present disclosure;

FIG. 21 is a perspective view of the anchor, an actuator, and a distal component of the inserter, according to an embodiment of the present disclosure;

FIG. 22A is a perspective view of an anchor and an actuator, according to an embodiment of the present disclosure;

FIG. 22B is an exploded perspective view of a toggle mechanism and an actuator 2002, according to an embodiment of the present disclosure;

FIG. 23 is an alternative perspective view of the system of FIG. 1A;

FIG. 24 is a perspective view of a system for providing compressive force, according to another embodiment of the present disclosure;

FIG. 25 is an alternative perspective view of the system of FIG. 24;

FIG. 26 is an alternative perspective view of the system of FIG. 24;

FIG. 27 is an antero-medial view of a partial tibia, talus, and general bone structure of a foot with a bone preparation tool positioned relative to the tibia and talus;

FIG. 28 is an antero-medial view of a partial tibia, talus, and general bone structure of a foot with a depth gauge positioned relative to the tibia, the talus;

FIG. 29 is an antero-medial view of the partial tibia, talus, and general bone structure of the foot with a bone preparation tool positioned relative to the tie tibia and talus;

FIG. 30 is a view of the anchor positioned for insertion into a tibiotalar joint through a hole extending obliquely through the tibia and talus along a single trajectory;

FIG. 31 is a close-up view of the anchor inserted into the tibiotalar joint through a hole extending obliquely through the tibia and talus, where the toggle mechanism is positioned to engage a portion of the talus;

FIG. 32A is an antero-medial view of a partial tibia, talus, and general bone structure of a foot with the system of FIG. 23 positioned to engage the anchor;

FIG. 32B is a close-up view of a toggle mechanism positioned for engaging a portion of the talus, according to an embodiment of the present disclosure;

FIG. 33A is an antero-medial view of a partial tibia, talus, and general bone structure of a foot with the system of FIG. 23 positioned to engage an anchor, according to another embodiment of the present disclosure;

FIG. 33B is a close-up view of a toggle mechanism positioned for engaging a portion of the talus, according to another embodiment of the present disclosure;

FIG. 34 is a view of the anchor extending obliquely through the tibia and talus, where the toggle mechanism is engaging a bone surface of the talus;

FIG. 35 is a view of the anchor extending obliquely through the tibia and talus, where the toggle mechanism is engaging a bone surface of the talus;

FIG. 36A is an antero-medial view of a partial tibia, talus, and general bone structure of a foot with the system of FIG. 23 positioned to engage and rotate the anchor, according to an embodiment of the present disclosure;

FIG. 36B is a close-up view of a toggle mechanism positioned to engage a portion of the talus, according to an embodiment of the present disclosure;

FIG. 37 is an antero-medial view of a partial tibia and talus with the system of FIG. 24 positioned to engage and rotate an anchor, according to another embodiment of the present disclosure;

FIG. 38 is an antero-medial view of a partial tibia and talus with the system of FIG. 24 positioned to engage and rotate the anchor, with the anchor positioned obliquely through a portion of the tibiotalar joint;

FIG. 39A is a close-up view of the anchor and toggle mechanism engaging the tibia and talus, according to an embodiment of the present disclosure;

FIG. 39B is a close-up view of the toggle mechanism of FIG. 39A;

FIG. 40 is an antero-medial view of a partial tibia, talus, and general bone structure of the foot, with a distal component of a distal end of the anchor engaging the tibia, according to an embodiment of the present disclosure;

FIG. 41A is an antero-medial view of a partial tibia, talus, and general bone structure of a foot with another system positioned to engage an anchor, according to another embodiment of the present disclosure;

FIG. 41B is a close-up view of a toggle mechanism in a retracted position and positioned to engage a portion of the talus, according to an embodiment of the present disclosure;

FIG. 42A is an anterior view of a partial tibia and talus with the system of FIG. 41A positioned to engage and apply an impact force to an anchor, according to another embodiment of the present disclosure;

FIG. 42B is an anterior view of a partial tibia and talus with the system of FIG. 41A positioned to engage an anchor during the application of an impact force;

FIG. 43 is a close-up view of an anchor in a retracted position relative to the tibia and talus, according to an embodiment of the present disclosure;

FIG. 44 is a close-up view of an anchor in a deployed position relative to the tibia and talus, according to an embodiment of the present disclosure;

FIG. 45 is a close-up view of a toggle mechanism engaging a bone surface, according to another embodiment of the present disclosure;

FIG. 46 is an anterior view of an anchor implanted between a talus and a tibia, with a proximal component abutting an exterior surface of the tibia and a distal component abutting an exterior surface of the talus;

FIG. 47 is an exploded anterior view of an anchor assembly, according to an embodiment of the present disclosure;

FIG. 48 is an anterior view of an assembled anchor of FIG. 47 in a retracted position;

FIG. 49 is an anterior view of the anchor of FIG. 48 and an actuator, according to an embodiment of the present disclosure;

FIG. 50 is an anterior view of the anchor of FIG. 48 positioned relative to a first bone portion surface and a second bone portion surface, according to an embodiment of the present disclosure;

FIG. 51 is an anterior view of the anchor of FIG. 48 in a deployed position after receipt of an actuation force relative to the actuator, according to an embodiment of the present disclosure;

FIG. 52 is an anterior view of the anchor of FIG. 48 after removal of the actuator, according to an embodiment of the present disclosure;

FIG. 53 is an anterior view of the anchor of FIG. 48 positioned to receive a rotational force, according to an embodiment of the present disclosure;

FIG. 54 is an anterior view of the anchor of FIG. 48 after receiving the rotational force, according to an embodiment of the present disclosure;

FIG. 55 is an anterior view of the anchor of FIG. 48, inserted into the talus and tibia via a retrograde approach; and

FIG. 56 is an anterior view of an anchor inserted into the talus and tibia such that the anchor terminates in the interior of the talus.

It is to be understood that the drawings are for purposes of illustrating the concepts of the disclosure and may not be drawn to scale. Furthermore, the drawings illustrate exemplary embodiments and do not represent limitations to the scope of the present disclosure.

### DETAILED DESCRIPTION

Exemplary embodiments of the present disclosure will be best understood by reference to the drawings, wherein like parts are designated by like numerals throughout. It will be readily understood that the components of the present disclosure, as generally described and illustrated in the Figures herein, could be arranged and designed in a wide variety of different configurations. Thus, the following more detailed description of the embodiments of the apparatus and method, as represented in the Figures, is not intended to limit the scope of the present disclosure, as claimed in this or any other application claiming priority to this application, but is merely representative of exemplary embodiments of the present disclosure.

Standard medical directions, planes of reference, and descriptive terminology are employed in this specification. For example, anterior means toward the front of the body. Posterior means toward the back of the body. Superior means toward the head. Inferior means toward the feet. Medial means toward the midline of the body. Lateral means away from the midline of the body. Axial means toward a central axis of the body. Abaxial means away from a central axis of the body. Ipsilateral means on the same side of the body. Contralateral means on the opposite side of the body. A sagittal plane divides a body into right and left portions. A midsagittal plane divides the body into bilaterally symmetric right and left halves. A coronal plane divides a body into anterior and posterior portions. A transverse plane divides a body into superior and inferior portions. These descriptive terms may be applied to an animate or inanimate body.

The phrases “connected to,” “coupled to” and “in communication with” refer to any form of interaction between two or more entities, including mechanical, electrical, magnetic, electromagnetic, fluid, and thermal interaction. Two components may be functionally coupled to each other even though they are not in direct contact with each other. The term “abutting” refers to items that are in direct physical contact with each other, although the items may not necessarily be attached together. The phrase “fluid communication” refers to two features that are connected such that a fluid within one feature is able to pass into the other feature.

The word “exemplary” is used herein to mean “serving as an example, instance, or illustration.” Any embodiment described herein as “exemplary” is not necessarily to be construed as preferred or advantageous over other embodiments. While the various aspects of the embodiments are presented in drawings, the drawings are not necessarily drawn to scale unless specifically indicated.

According to some embodiments, a bone compression system is disclosed. The system may optionally be used by itself as a method to compress a fracture, or joint space to achieve a fusion.

The system can also be used in conjunction with a bone fixation system such as a plate system or an intramedullary rod or nail system. The system used in these indications may be uniquely suited to reduce a fracture or articular surface of a joint, unlike the current locking screws and non-locking screws that are used in plate and intramedullary rod/nail systems that are not able to reduce a fracture or joint surface.

The system may be inserted through one of the holes in a non-locking or locking bone plate. The system in an intramedullary rod system may be inserted through a hole in the rod/nail. The system may be used in conjunction with other locking and/or non-locking screws for the plate and intramedullary rod systems. Examples of bones or joints at which this could be utilized with plate or rod system include, but are not limited to, the proximal and distal tibia, the proximal and distal femur, and the proximal and distal humerus. Exemplary bones or joints at which this could be utilized with an intramedullary rod system include, but are not limited to, a humeral rod, a tibial rod, and a femoral rod, including a supracondylar rod for distal femur fractures. The system may additionally or alternatively be included as part of an intramedullary rod or plate system that is utilized for peri prosthetic fractures after arthroplasty.

The system may be used in conjunction with a spacer, such as a bone support implant. Spacer members may include openings for screw fixation, and/or anterior or lateral plates for additional stabilization and screw fixation. The system disclosed herein may pass through a hole formed in the spacer. Attachment features such as flanges, bolt and/or screw apertures, and instrument connection features may be included on any of the spacing members. The spacing members disclosed herein may include any suitable biocompatible material including, but not limited to: plastics including PEEK, carbon fiber reinforced PEEK, glass filled PEEK, UHMWPE, polyurethane, PEKK, and PET; metals and metal alloys including titanium, titanium alloys, stainless steel, cobalt chrome, tantalum, and barium; ceramics including those including alumina, zirconia, zirconium, and silicon nitride; pyrolitic carbon; and coatings including hydroxyapatite, porous titanium, silicon nitride, titanium carbide, and titanium nitride.

The system may have a toggle anchor (e.g., toggle mechanism) that is attached to a screw distal portion of the system. The system may have a larger proximal portion that captures a smaller distal portion. The distal portion may be insertable into the proximal portion and translatably adjustable within the proximal portion. The proximal portion of the system may be larger in diameter than the distal portion of the system. This specific design therefore may allow the system to achieve a ‘lag’ effect when compressing a fracture or compressing a joint surface for fusion.

The system may be cannulated throughout the whole system, including both proximal and distal portions of the system. This cannulation may facilitate deployment of the toggle. Once the system has been deployed, the two suture ends that are present on the proximal part of the system can be then tied and knotted together to prevent backing out or loosening of the proximal portion of the system.

A curved washer may be utilized with the system and may be made of various metals including stainless steel, titanium, and Nitinol. The washer, when utilized as part of the system, may provide dynamic continued compression and prevent backing out and/or loosening of the proximal portion of the system.

The toggle may have eyelets to allow passage of a suture that is utilized for deployment of the toggle. The toggle may additionally or alternatively have a keel or fin that allows for additional purchase on the cortical bone and prevent rotation of the system when compression is applied.

The distal portion of the system may have keels and/or fins that allow for additional stability in the distal part of the bone or opposing joint surface, thereby preventing rotation when compression is applied when the proximal portion of the system is engaged.

The device may have dynamic compression ability. The curved washer can add dynamic compression but also making the toggle a Nitinol element can create dynamic compression by shape setting where it is domed, when relaxed, but then flattens as compression is applied. This may create an inbuilt preload to keep the bones compressed. It may also help to add pre-loads or pre-stress during formation of the toggle, thereby providing additional anti-backing features.

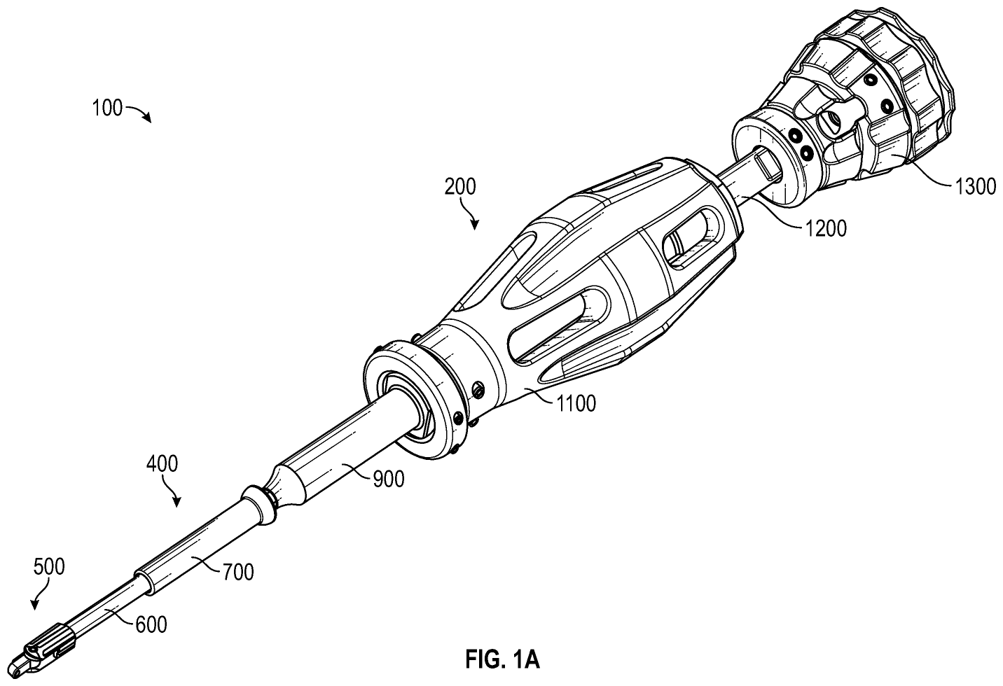

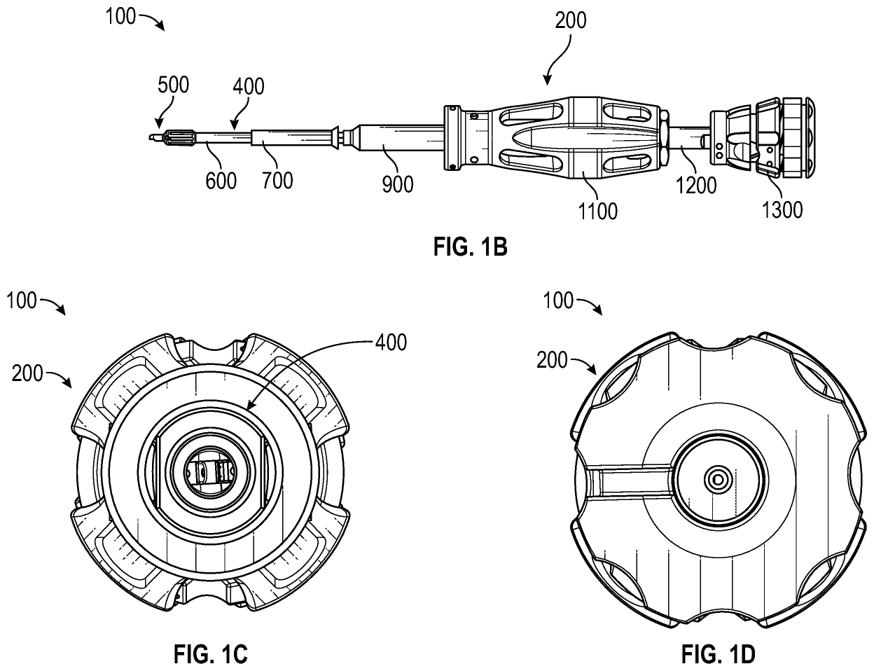

FIGS. 1A, 1B, 1C, and 1D illustrates perspective, side elevation, front elevation, and rear elevation views, respectively, of a system 100 for providing compressive force relative to a first bone portion and a second bone portion, according to an embodiment of the present disclosure. The first bone portion and the second bone portion may be any two bones of the body, or any two fragments of a single bone. Bones of the foot will be referenced below by way of example.

Generally, the system 100 may include an inserter 200 and an anchor 400. The anchor 400 may have a proximal component 700 and a distal component 600, which may be translatably coupled to each other, to permit adjustment of the length of the anchor 400 to provide bone compression after implantation, as will be described below. The distal component 600 may have a toggle mechanism 500 that is pivotably connected to the remainder of the distal component 600.

The inserter 200 may have a distal extension 900 that removably connects the distal component 600 to the proximal component 700. The inserter 200 may include a first grip member 1100, which may be connected to a shaft member 1200. The shaft member 1200 may be connected to a second grip member 1300 such that a cannulation runs through the first grip member 1100, the shaft member 1200, and the second grip member 1300, providing access to the anchor 400 from proximal to the second grip member 1300.

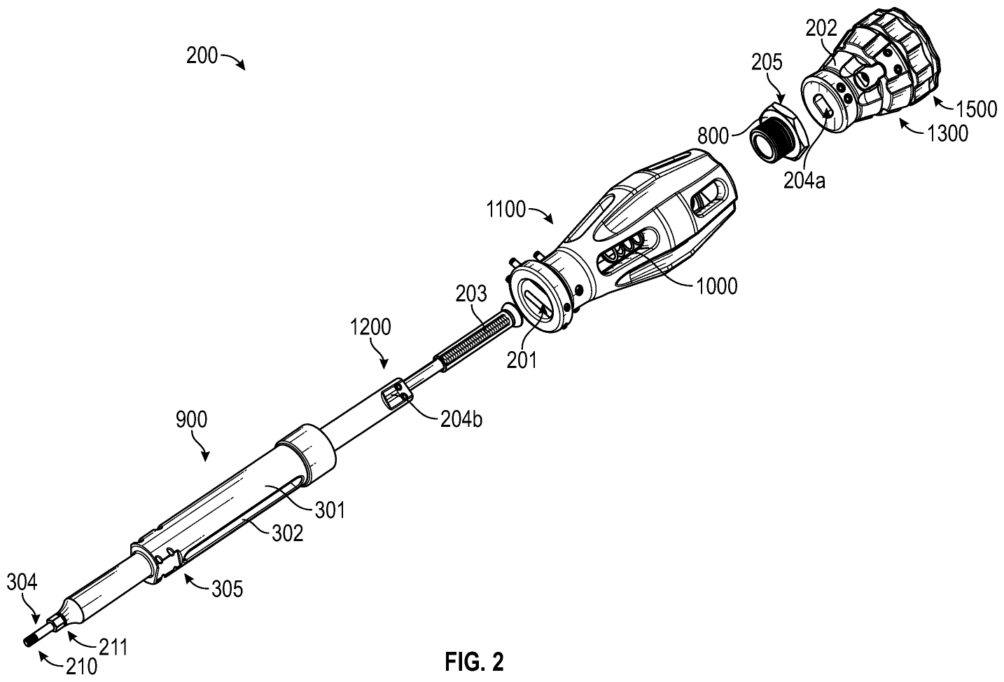

FIG. 2 illustrates an exploded view of the inserter 200, according to an embodiment of the present disclosure. As mentioned above, the components of the inserter 200 may be cannulated to facilitate passage of an actuator (discussed below) through the components of the assembly.

The first grip member 1100 may have an aperture 201 configured to transfer a first rotational force from a grip (e.g., knurled surface) of the first grip member 1100 to the anchor 400. The transfer of the first rotational force may rotate a drive shaft 301 of the inserter 200. The rotation of the drive shaft 301 may transfer the first rotational force to a hexagonal end 211 of a force-toggling tip 304.

The inserter 200 may include additional apertures formed in various components of the inserter 200. For example, the drive shaft 301 may include an elongated aperture 302, which may provide access to a biasing member 1000 through a corresponding elongated aperture in the first grip member 1100. Accessing the biasing member 1000 may enable a user to more easily determine if/when the biasing member 1000 may need to be replaced.

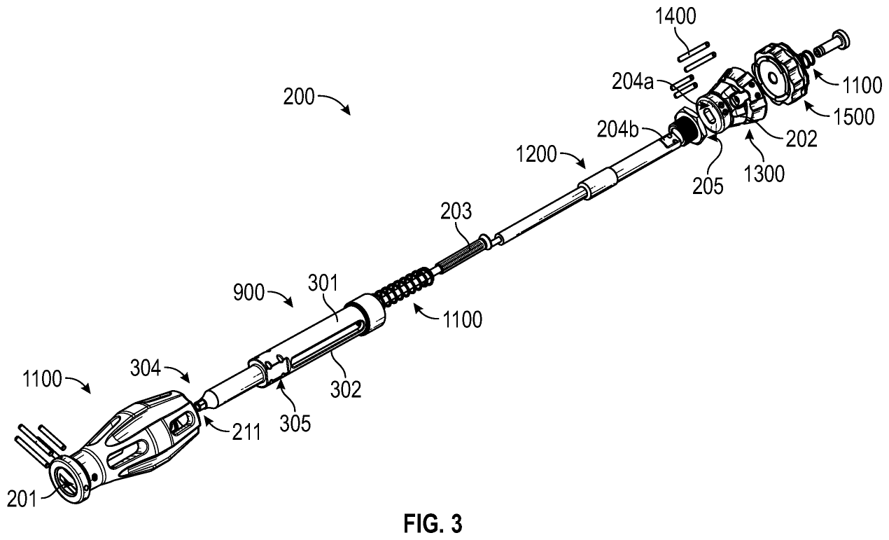

The second grip member 1300 may have a handle 202 with a grip (e.g., knurled surface) configured to receive a second rotational force to rotate a drive feature of the shaft member 1200. For example, an opening with a drive seat 204a, having pins 1400 (FIG. 3) within the opening may be configured to engage flat drive surfaces 204b of the shaft member 1200. The shaft member 1200 may be connected to a bored/threaded female bolt member that has internal threads 203. The internal threads 203 may be connected to the flat drive surfaces 204b of the shaft member 1200 by, for example, a hollow, double-end threaded bolt. The flat drive surfaces 204b may receive the second rotational force applied to the handle 202 via the pins 1400 of the drive seat 204a, thereby transferring that second rotational force to the shaft member 1200. The shaft member 1200 may transfer the second rotational force to a threaded end 210 of the force-toggling tip 304.

The first grip member 1100 may include a threaded component 800 that has flat surfaces 205. At least one of the flat surfaces 205 may include a compaction surface. The flat surfaces 205 may abut a proximal end of the first grip member 1100. The threads of the threaded component 800 may engage corresponding threads of a female proximal end of the drive shaft 301.

The first grip member 1100 may further include one or more biasing members such as a biasing member 1000, which may be a helical spring. In alternative embodiments, other biasing members may be used, such as torsional springs, leaf springs, pneumatic cylinders, and/or the like. The biasing member 1000 may allow a third grip member 1500 to receive a compressive force to compress the biasing member 1000. The compressive force applied to the third grip member 1500, together with the application of the second rotational force may rotate each of the second grip member 1300, the third grip member 1500, and the shaft member 1200. The rotation of the shaft member 1200 may transfer the second rotational force to the threaded end 210 of the inserter 200. The distal component 600 may have internal threads that engage the threaded end 210 to attach and/or remove the distal component from the inserter 200.

In some embodiments, the pins 1400 may be inserted through the handle 202 into holes in the proximal end of the shaft member 1200 to provide stability and further facilitate transfer of the second rotational force to the shaft member 1200.

The third grip member 1500 (FIG. 15) may be connected to, or may be in rotational contact with, the female bolt having the internal threads 203. A portion of this female bolt may be within the cannulated portion of the handle 202. The third grip member 1500 may include a head or knob, which may be biased, a grip (e.g., knurled surface), and a pawled surface 1502. In some embodiments, the head is biased by the biasing member 1000. In other embodiments, the head is biased by a second biasing member (not shown) separate from the biasing member 1000.

In some embodiments, when the biasing member 1000 is compressed, it causes the pawled surface of the third grip member 1500 to engage the handle 202. Additionally, when the biasing member 1000 is compressed, the third grip member 1500 may be used, together with the handle 202, to rotate the threaded end 210 of the force-toggling tip 304. In some embodiments, when the biasing member 1000 is decompressed, the pawled surface is disengaged from the proximal surface of the handle 202. Additionally, when the biasing member 1000 is decompressed, the third grip member 1500 may be used to twist, tie, and/or secure an actuator 2002 (discussed in greater detail below). In yet other embodiments, the third grip member 1500 may be configured such that a knob (not shown) of the third grip member 1500 must be compressed in order to rotate the first grip member 1100, the shaft member 1200, and/or the drive shaft 301. In these embodiments, the third grip member 1500 may ensure that compression is applied during rotation of the anchor 400 and/or as a safety mechanism for the system 100.

The force-toggling tip 304 may toggle between application of force to the threaded end 210 and application of force to the hexagonal end 211 of the inserter 200. In some embodiments, the toggling feature of the force-toggling tip 304 may allow the inserter to be used for multiple interbody implants, such as the anchor 400, a bone screw, a rod/nail system, another type of fusion nail, and combinations thereof. Although the ends of the force-toggling tip 304 are described as a hexagonal end 211 and a threaded end 210, other force-transferring features may be interchangeably included in the force-toggling tip 304, including but not limited to, an Allen-wrench end, a Phillips end, an octagonal end, a pentagonal end, a star-shaped end, a Torx fitting, and/or combinations thereof.

FIG. 3 illustrates an alternative exploded view of the inserter 200 of FIG. 2. In this alternative view, the pins 1400 are shown removed from the handle 202 and from the first grip member 1100.

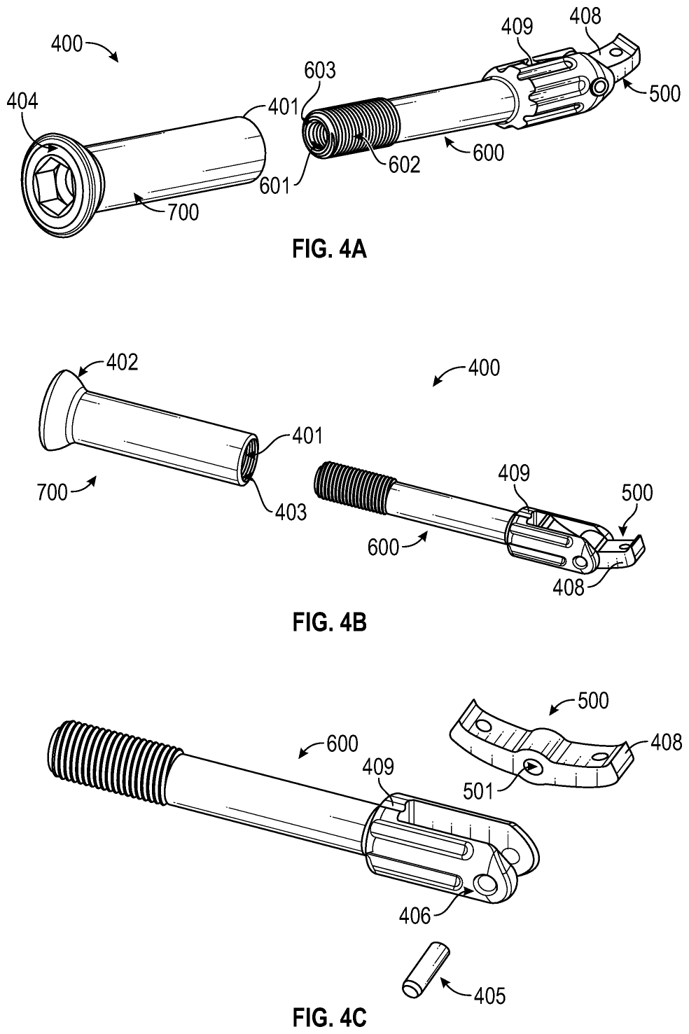

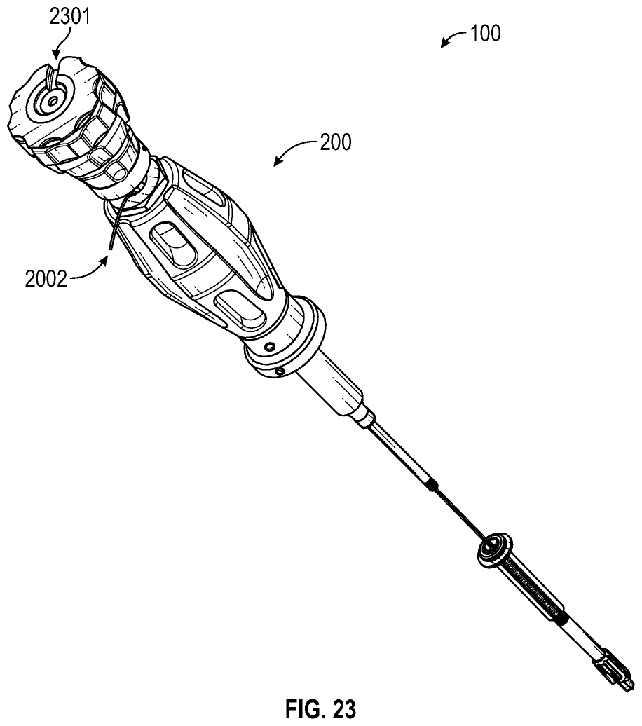

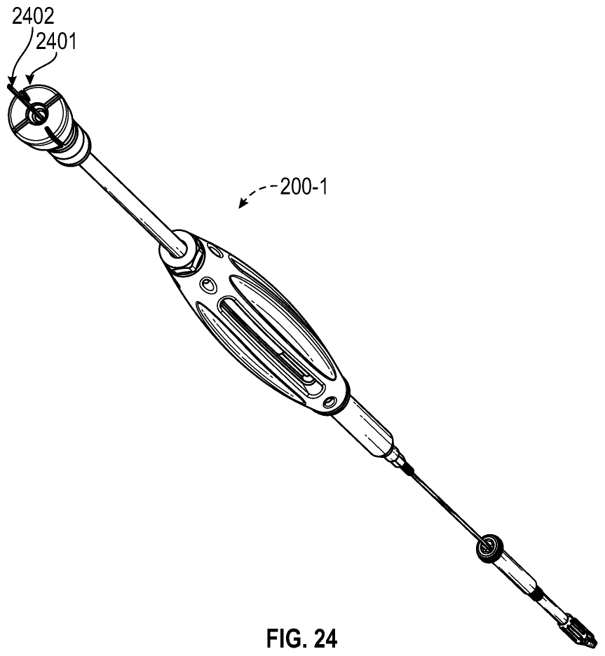

As disclosed herein, the systems, devices, and methods of the present disclosure may include fixation systems (e.g., FIGS. 23 and 24) that may include implants (e.g., FIGS. 4A through 4C, 16, and 17) for fusing a joint or repairing a bone fracture between a first bone portion 2701 and a second bone portion 2702. In some embodiments, the systems may be used in a tibial osteotomy, and in other embodiments, the systems may be used in ankle arthrodesis.

In some embodiments, the anchor 400 may include the proximal component 700, the distal component 600, the toggle mechanism 500, and a washer 1600. The toggle mechanism 500 may be deployable to couple the distal component 600 to the second bone portion 2702 to facilitate application of compression between the first bone portion 2701 and the second bone portion 2702. The toggle mechanism 500 may be formed at least partially of a flexible superelastic material configured to maintain the compression force between the first bone portion 2701 and the second bone portion 2702. The proximal component 700 may be a cannulated female coupling member with a head 402 and internal threads 403 along a length of the proximal component 700. The head 402 may include a rounded, beveled, and/or smooth surface for engaging a bone surface perimeter.

The proximal component 700 may have a drive feature (e.g., a hexagonal socket of the head 402) to coaxially accommodate the inserter 200. The distal component 600 may be a hollow/cannulated male member (e.g., distal component 600) with a proximal end 601 with proximal external threads 602 designed to coaxially accommodate the internal threads 403 of the proximal component 700.

In addition to the toggle mechanism 500, the distal component 600 may further include features to accommodate and secure the toggle mechanism 500 at the distal end of the distal component 600. The distal component 600 may have a distal coupling feature (e.g., housing, pin 405, and/or pin hole 406) that allows for rotation and/or pivoting of the toggle mechanism 500 via a pivot hole 501. The distal component 600 may have features, such as internal threads 603, to accommodate and secure a threaded end 210 of the force-toggling tip 304 of the inserter (e.g., inserter 200) in the internal threads 603. The toggle mechanism 500 may have an elongated toggle element with features (e.g., holes 1706 and/or holes 2001) that allow for an actuator 2002 (for example, one or more sutures) to pass through the elongated toggle element. The sutures may be laced and/or threaded through the elongated toggle element in a manner that provides the ability to flip the toggle mechanism 500 in situ through the cannulated components of the anchor 400 and the inserter 200. Alternatively, the toggle mechanism 500 can also be flipped by using an alternative actuator such as a rod and/or wire that extends through the proximal component 700 and/or the distal component 600 to deploy the toggle mechanism 500 in situ.

The toggle mechanism 500 of the distal component 600 may be coupled to the remainder of the distal component 600. The toggle mechanism 500 may incorporate a material that is sufficiently flexible to provide a spring force against the second bone portion 2702 in the deployed configuration (e.g., see FIGS. 53 and 54). In some embodiments, an elongated member 408 and/or 508 may be sized relative to a housing 409 to allow for 360° rotation relative to the housing 409. In alternative embodiments, the distal component 600 has features to restrict a motion of the toggle mechanism 500, such that its motion may be in a single direction (i.e., clockwise or counterclockwise), thereby preventing it from moving in an opposite direction and/or or spinning in a 360° rotation. For example, the size of an opening in the housing that receives the elongated member 408 may be shorter in length than a length of the elongated member 408, thereby preventing the elongated member 408 from rotating 360°. By way of another example, pins, detents, catches, or similar structures may be used to prevent a full 360° rotation of the elongated member 408.

FIGS. 4A through 4C illustrate views of the anchor 400 of the system 100. The anchor 400 may include the distal component 600 and the proximal component 700 as mentioned previously. The proximal component 700 may include a hollow, cannulated, and/or bored female coupling member 401 with internal threads 403 and a drive seat 404, such as a hexagonally shaped interior head socket. Although the drive seat 404 is depicted as hexagonal, other drive seat shapes are contemplated and included herein, including but not limited to star, square, cross (i.e., Phillips), pentagonal, octagonal, and/or combinations thereof.

The anchor 400 may further include coupling features to secure the toggle mechanism 500 to the remainder of the distal component 600. For example, a pin 405 and a pin hole 406 may secure an elongated member 408 of the toggle mechanism 500 to the remainder of the distal component 600. Threads may secure a housing 409 of the toggle mechanism 500 to the remainder of the distal component 600. In other embodiments, other securing features may be used, such as one or more ball detents, washers, partial thread bolts (e.g., shoulder bolt), screws, and/or combinations thereof.

In some embodiments, the housing 409 includes a knurled surface, keels, fins, texturing, or another protruding surface to engage interior bone surfaces of a bore hole through a first bone portion and/or a second bone portion. In other embodiments, the elongated member 408 is a curved member with keels, fins, or other protruding features to help engage bone surfaces beyond a diameter of the bore hole through the first bone portion and/or the second bone portion. The elongated member 408 may be formed of a material that has dynamic compression abilities (discussed in greater detail below).

It is appreciated that in this and other embodiments of the invention, the relative footprint, lengths, widths, heights and shapes of the proximal component 700 and the distal component may vary as needed to fit patient anatomy and/or desired correction. For example, a medial end may be longer than a lateral end or the length of the distal and proximal components may vary as needed. Similarly, the specific curvatures of the bone-engaging and bearing surfaces may vary to suit patient anatomy and mobility needs. For example, the shoulder of the proximal component configured to engage a tibial bone surface may be curved, and the elongated member of the toggle mechanism configured to engage the talar bone surface may also be curved. In other embodiments, at least one of the shoulder and the elongated member may be planar. In some embodiments, the bone-facing surfaces of the shoulder and the elongated member may include coatings or treatments including but not limited to: hydroxyapatite, porous titanium, silicon nitride, titanium carbide, and titanium nitride.

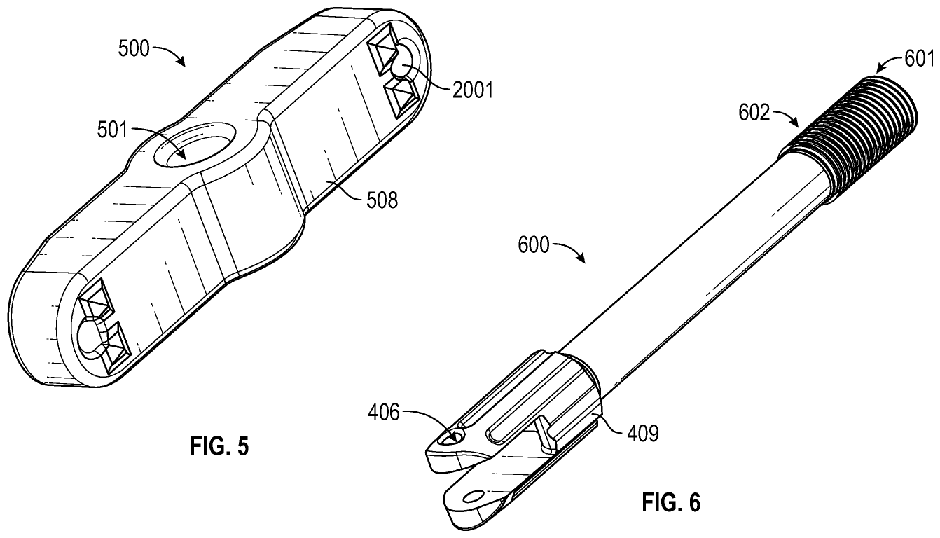

FIG. 5 illustrates an exploded view of a moveable portion of the toggle mechanism 500, according to an embodiment of the present disclosure. The moveable portion may include an elongated member 508 of the toggle mechanism 500, which may be formed to include a pivot hole 501. In some embodiments, the pivot hole 501 may be aligned with the pin hole 406 for receiving the pin 405 and securing the elongated member 508, instead of the elongated member 408 (e.g., one being interchangeable with the other). The elongated member 508 may have the same or a different size/shape than the elongated member 408. For example, the elongated member 508 may have more, or less, curvature than the elongated member 408. The elongated member 508 may comprise a material having elastic, superelastic, “shape memory”, pseudoelastic, or dynamic compression ability properties. For example, the elongated member 508 may have a material composition that includes a titanium-nickel allow (e.g., nitinol), a medical-grade cobalt-chromium alloy (e.g., Cr—Al—Si or CCAS, such as Co₅₁Cr₃₄Al₇Si₈), and combinations thereof. These properties of the elongated member 508, its shape, or how its surface is formed (e.g., with fins, keels, or other protrusions) in combination or individually, may provide a dynamic compressibility to the toggle mechanism 500. In other embodiments, there may be an inbuilt preload stress provided to the elongated member 508 during the formation of the elongated member 508 that contributes to the dynamic compressibility of the toggle mechanism 500.

In some embodiments, teeth, ribbing, protrusions, and/or texturing may be formed on a proximal and/or medial surface of the elongated member to provide a gripping effect and/or contribute to the dynamic compressibility of the toggle mechanism 500. In other embodiments, the protrusions may be formed on two or more opposing surfaces of the toggle mechanism 500, allowing the toggle mechanism to be rotated in either direction so that either the proximal or the distal side may be selected to abut an adjacent bone surface. In some embodiments, each side of the two or more opposing sides may provide a different dynamic compressibility, depending on the side that is selected to abut the bone surface.

Apertures or holes 2001 (discussed in greater detail below), may also be formed in the elongated member 508 to receive an actuator, such as one or more sutures. The actuator may be inserted according to a lacing setup or a threading pattern. In some embodiments, different lacing setups and/or threading patterns may be used to rotate or move the toggle mechanism between a retracted position and a deployed position, or according to a first direction and/or a second direction.

FIG. 6 illustrates an alternative perspective view of the distal component 600 of the anchor 400. In some embodiments, the distal component 600 may include a cylindrical threaded bolt (e.g., compression bolt) that engages with the female portion of the proximal component 700, having the housing 409 integrally formed thereon. For example, the pin hole 406 may be formed in the opposite end (non-threaded end) of the bolt together with an opening to receive an elongated member 408 or 508. In other embodiments, the distal component 600 may be a double-end bolt that has the housing 409 and the pin hole 406 attached to a distal end of the double-end bolt.

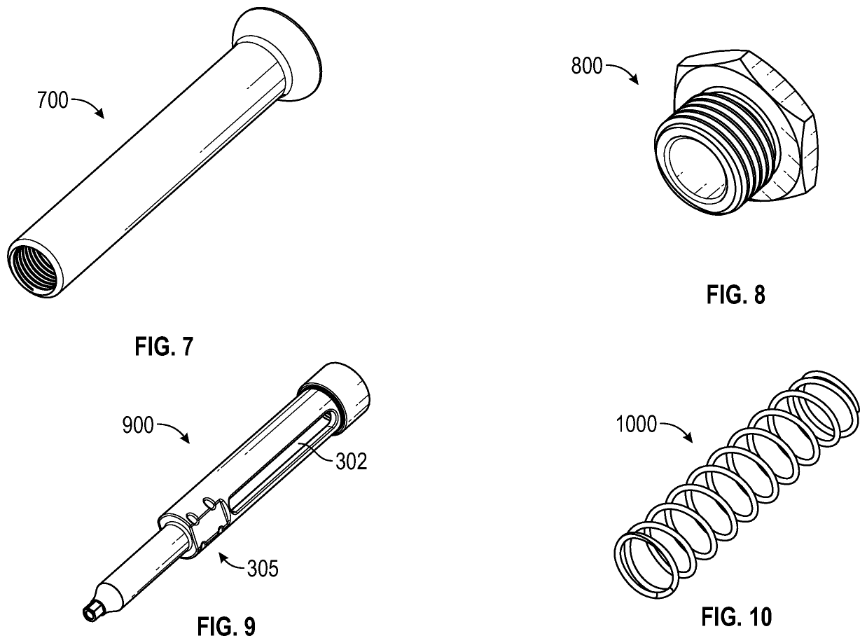

FIG. 7 is a perspective view of the proximal component 700 of the anchor 400. The shape or exterior of the proximal component 700 may be cylindrical, similar to that of a socket head bolt. However, rather than having external threads, the proximal component 700 may include internal threads similar to a coupling nut or threaded sleeve.

FIG. 8 is a perspective view of the threaded component 800 of the inserter 200. The threaded component 800 may comprise a cannulated hex head bolt, impact bolt, and/or compression bolt with a load-bearing surface (e.g., flat surface 205). Other head shapes, such as octagonal, pentagonal, or shapes with more/less surfaces are contemplated and included herein.

FIG. 9 illustrates a perspective view of a distal extension 900 of the inserter 200. The distal extension 900 may include an elongated aperture 302 and flat drive surfaces 305 for accessing, engaging, and/or disengaging a portion of the first grip member 1100. For example, in some embodiments, the elongated aperture 302 may engage a protruding internal portion of the grip member for stable housing of the distal extension 900 within the first grip member 1100.

The distal extension 900 may include apertures formed perpendicular to a longitudinal length of the cylindrical body. These apertures may be used to receive the pins 1400 for securing the distal extension 900 relative to the first grip member 1100.

The distal extension 900 may include a female, internally threaded proximal end. The female proximal end may accommodate the external threads of the threaded component 800.

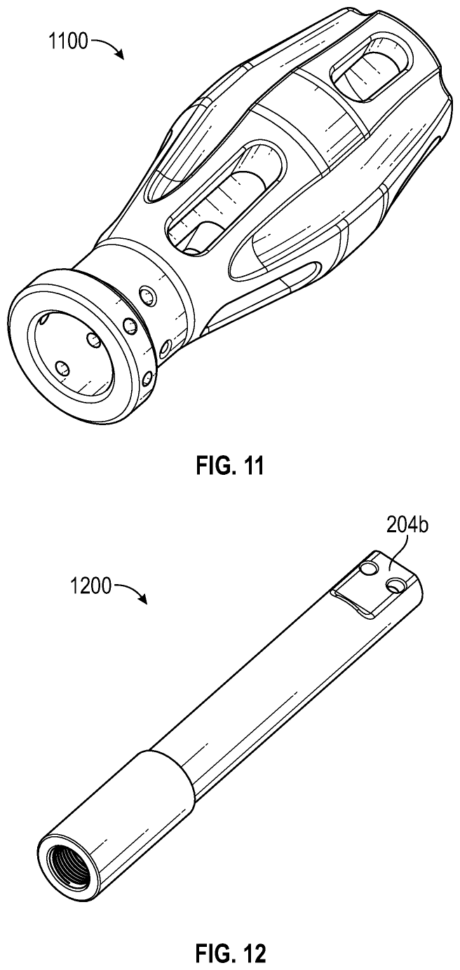

FIG. 10 illustrates a perspective view of a biasing member 1000 of the inserter 200. Although the biasing member 1000 is depicted as a helical coil spring, the first grip member 1100 may be formed to include a different type of biasing member 1000, such as a hollow Belleville spring, variable-rate spring, flat spring, CNC machined spring, molded spring, gas spring, and/or combinations thereof.

FIG. 11 illustrates a perspective view of the first grip member 1100 of the inserter 200. The first grip member 1100 may include a housing, a knurled and/or textured surface, internal housing or seat structures to seat components, such as a shoulder of the drive shaft 301, and multiple apertures/holes, such as the holes for receiving pins 1400.

FIG. 12 illustrates a perspective view of a hollow, cannulated shaft member 1200 of the inserter 200. The shaft member 1200 includes flat drive surfaces 204b that engage with the drive feature 204 of the handle 202. A proximal end of the shaft member 1200 may include holes for receiving pins 1400. A distal end of the shaft member 1200 may include internal threads (not shown) for engaging a proximal threaded end. The proximal threaded end engaged by the distal end of the shaft member 1200 may be one end of a hollow, double end threaded bolt that comprises a portion of the third distal component or a portion of the threaded end 210 of the force-toggling tip 304.

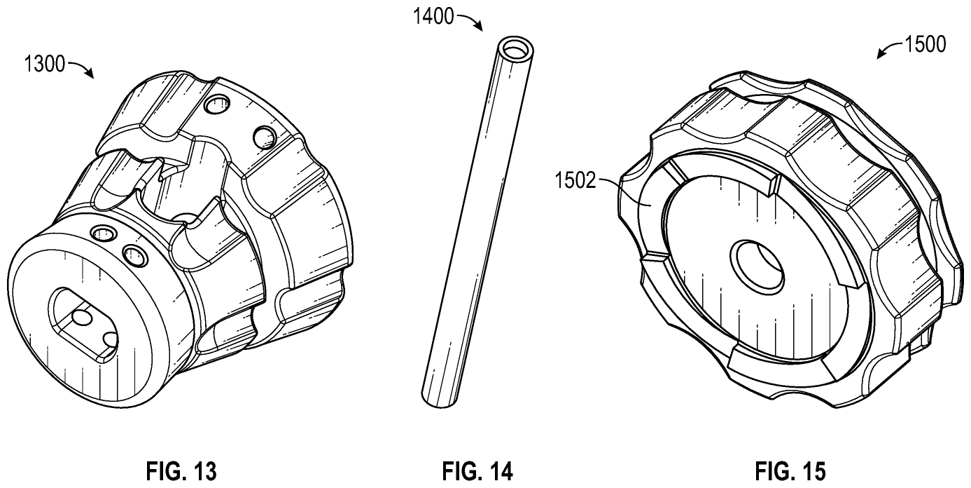

FIG. 13 illustrates a perspective view of the second grip member 1300 of the inserter 200. The second grip member 1300 may include a housing, a grip (e.g., knurled and/or textured surface), internal housing to seat a female internally threaded bolt having internal threads 203, and holes for receiving pins 1400.

FIG. 14 illustrates a perspective view of a pin 1400 of the inserter 200. The pins 1400 may be received within holes formed in a grip member and may be constructed of a material to receive forces (e.g., compression and/or impact forces) and/or may comprise a structurally reinforced material. For example, the pins 1400 may comprise stainless steel, tungsten, titanium, nickel, cobalt, chromium, or combinations thereof. Structural features, such as webbing, ribbing, pre-stressing, or combinations thereof may ensure the pins may be able to receive repeated compression and/or compacting forces.

FIG. 15 illustrates a perspective view of the third grip member 1500 of the inserter 200. A proximal surface (FIG. 1D) may include a depression for receiving a head of a hollow female bolt. A distal surface may include a pawled surface 1502 that engages with one or more protrusions (e.g., corresponding pawled surface) on the proximal surface of the second grip member 1300, ensuring that rotation occurs in a desired direction when compressive force is received at the proximal end of the third grip member 1500.

In some embodiments, components of the system 100 are described as cylindrical. In other embodiments, certain components of the system 100 may be non-cylindrical. For example, the first grip member 1100, the second grip member 1300, the third grip member 1500, and/or the pins 1400 may be non-cylindrical.

Suitable materials for the hardware disclosed herein, including but not limited to compression bolts, sleeves, screws, washers, flanges, toggles, tabs, nuts, and anchors, may include any biocompatible metals and metal alloys, including titanium/titanium alloys, stainless steel, cobalt chrome, tantalum, and barium.

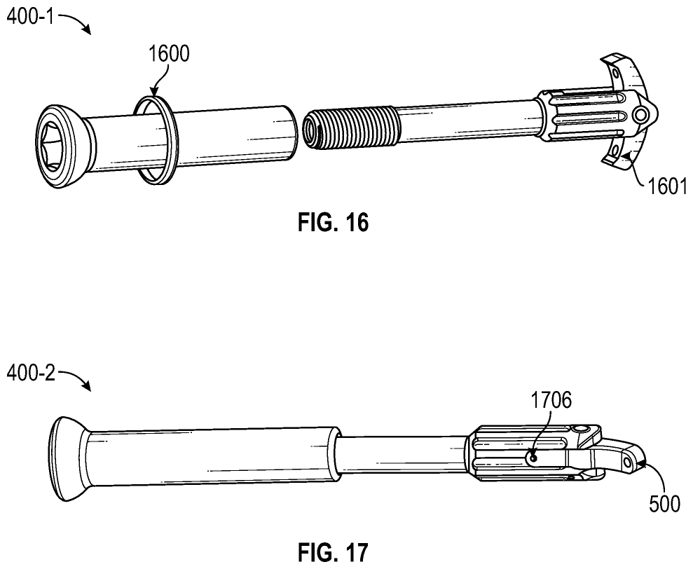

FIG. 16 is a perspective view of an anchor 400-1, according to an alternative embodiment. The anchor 400-1 may be shaped, sized, or geometrically configured differently than the anchor 400. For example, the anchor 400-1 may be larger or smaller in diameter and/or length than the anchor 400. By way of another example, the curved surface 1601 of the elongated member of the toggle mechanism may include hooked protrusions at each end of the curved surface 1601 and/or be made of a different material than the elongated member 408 or the elongated member 508. The radius of curvature of the curved surface 1601 may be different (e.g., larger or smaller) than the radius of curvature of the elongated member 408 or the elongated member 508. By way of yet another example, the anchor 400-1 may be different from the anchor 400 by the addition of one or more structural features. For instance, the anchor 400-1 may include a washer 1600, which may be a curved compression washer. The washer 1600 may add a dynamic compression effect to the system 100. The washer 1600 may be formed partially or entirely of a compressible and/or elastic material such as Nitinol, CCAS, or another elastic or superelastic material. The washer 1600 may assume at least two different positions. For example, the washer 1600 may assume a relaxed, or unbiased position, where it has a domed shape. The washer 1600 may also assume a stressed, or biased position, where it is flattened after compression is applied.

FIG. 17 illustrates a perspective view of an anchor 400-2, according to another alternative embodiment. The anchor 400-2 may be similar to the anchor 400 and/or the anchor 400-1, except that in some embodiments, one or more features may be removed and/or interchanged from anchor 400-2 relative to the other anchor members. For example, anchor 400-2 may not include a washer 1600 and the toggle mechanism 500 may be removable such that the elongated member may be interchangeable with various other shaped/sized elongated members and/or toggle mechanisms. A surface of the elongated member of the anchor 400-2 may be smooth (e.g., formed without any protrusions, keels, or fins). A hole 1706 may have a different shape and/or size than a hole in the toggle mechanism of the anchor 400. In other embodiments, a greater or lesser number of holes 1706 may be included in the elongated member of the toggle mechanism 500 of anchor 400-2 to accommodate a greater and/or lesser number, the same and/or different sized, or the same and/or different lacing setup or threading pattern of actuators. In yet other embodiments, the housing of the toggle mechanism may be shaped and/or sized to prevent a 360° rotation of the elongated member of the toggle mechanism 500.

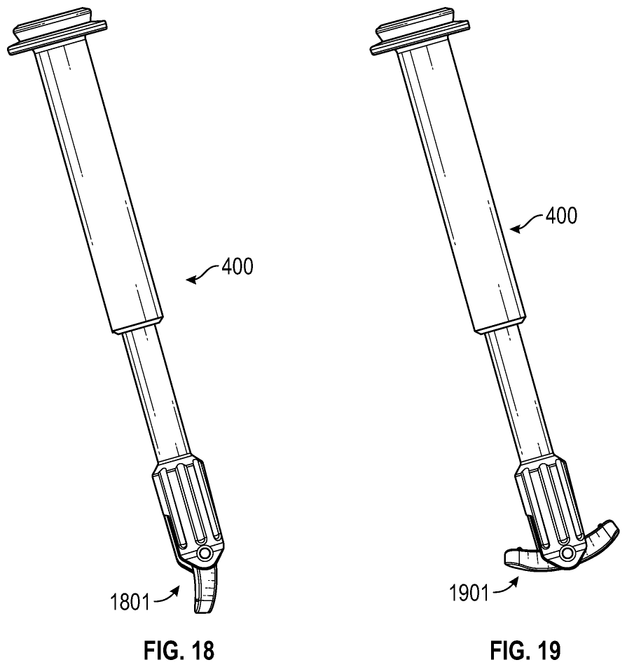

FIG. 18 illustrates a perspective view of a retracted position 1801 of a toggle mechanism, which may be inserted through a bore hole in a bone with minimal resistance. In some embodiments, the retracted position assumes a smaller width than that of the proximal component of the anchor 400. FIG. 19 illustrates a perspective view of a deployed position 1901 of a toggle mechanism of an anchor 400.

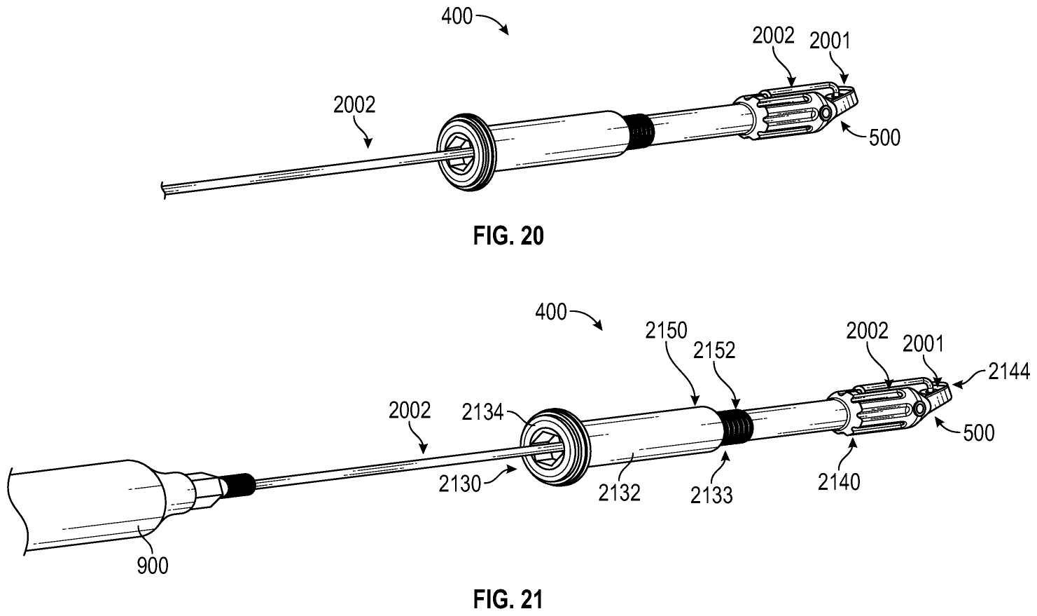

FIG. 20 illustrates a perspective view of the anchor 400 and an actuator 2002, according to an embodiment of the present disclosure. The anchor 400 and actuator 2002 may work in conjunction to provide a compressive force between bone surfaces. For example, the distal end of the anchor 400 may include the toggle mechanism 500 with one or more actuator holes, such as hole 1706, and an actuator 2002 threaded through the actuator holes. The engagement of the actuator with the actuator holes may provide the movement of the toggle mechanism 500 between the retracted and deployed positions (e.g., 1801 and 1901).

In some embodiments, the actuator 2002 may include one or more sutures. In other embodiments, the actuator 2002 may include an actuatable ball detent, retractable pin, releasable pre-biased member, wire, string, threads (e.g., sewing threads), cable, or combinations thereof.

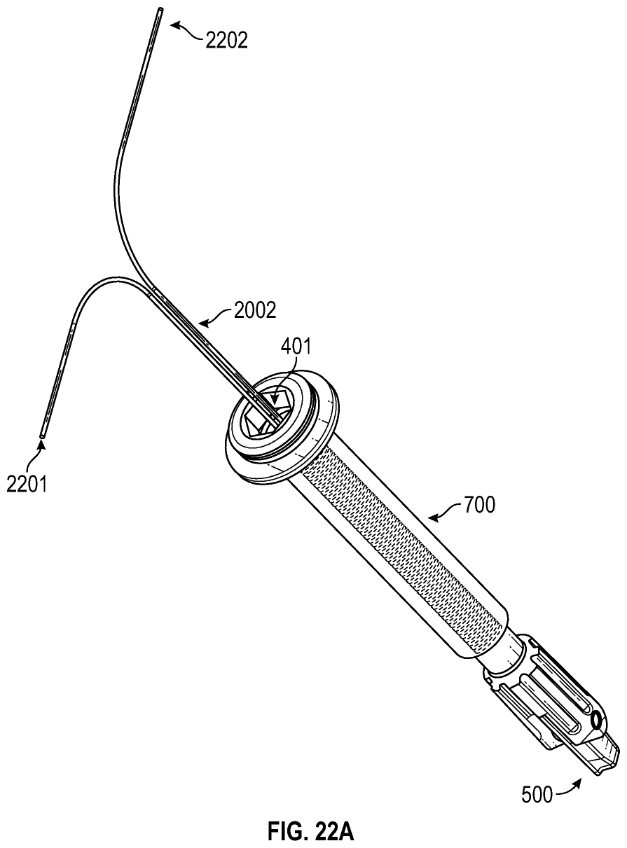

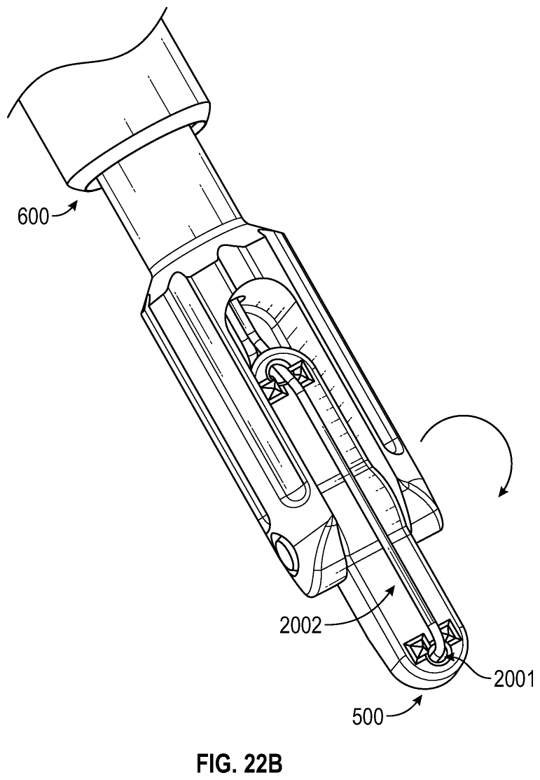

FIG. 21 is a perspective view of a partial assembly of the system 100. The partial assembly may include the anchor 400, the actuator 2002, and the distal extension 900 of the inserter. The anchor 400 may be sized and shaped to be insertable into a hole formed through at least part of a first bone portion (e.g., tibia) and a second bone portion (e.g., talus). The partial assembly may further include a proximal component proximal end 2130, a proximal component 2132, and a head 2134 of the proximal component 2132. The proximal component 2132 may further include a proximal component distal end with a proximal coupling feature 2133, such as internal threads (FIG. 22A).

The partial assembly may also include the toggle mechanism 500 attached to the distal component 2140 at the distal component distal end 2144. The distal component proximal end 2150 may include a distal component coupling feature 2152, such as external threads, to translatably engage the proximal coupling feature 2133 of the proximal component 2132.

FIG. 22A illustrates a perspective view of an anchor and an actuator 2002. The internal threads of the proximal component 700 are also depicted in broken lines. The actuator 2002 may include a first end 2201 and a second end 2202. Actuation (e.g., pulling, tugging, etc.) of the first end 2201 or the second end 2202 may rotate the elongated member of the toggle mechanism 500 in a first direction or a second direction into the deployed position 1901, depending on the engagement and/or path of the actuator 2002 relative to the holes 1706 and/or holes 2001.

FIG. 22B illustrates an exploded perspective view of the toggle mechanism 500 and the actuator 2002, according to an embodiment of the present disclosure. The actuator 2002 may engage the elongated member of the toggle mechanism 500 through the holes 2001 at a distal end of the distal component 600 of an anchor member, such as anchor 400. The actuator 2002 may have a flexible structure to engage the holes 2001 according to one or more different paths, patterns, and/or threading configurations.

FIG. 23 illustrates an alternative perspective view of the system 100 of FIG. 1A. The inserter 200 may be offset from the anchor 400 to depict the actuator 2002 connecting the inserter 200 and the anchor 400. An end of the actuator 2002 may pass through multiple components of the cannulated components of the system 100. In some embodiments, the end of the actuator 2002 exits the proximal end of the first grip member 1100 for actuating the toggle mechanism. In other embodiments, the end of the actuator 2002 exits a notch 2301 in the head of the third grip member 1500. In yet other embodiments, one or more notches 2301, the head/knob of the third grip member 1500, and a female bolt of the third grip member 1500 may be used to engage, twist, tie or otherwise interact with ends of the actuator 2002 to deploy the toggle mechanism, retract slack in the actuator (e.g., by twisting the knob), and/or secure the actuator after compression has been applied to a first bone portion and/or a second bone portion using the anchor 400.

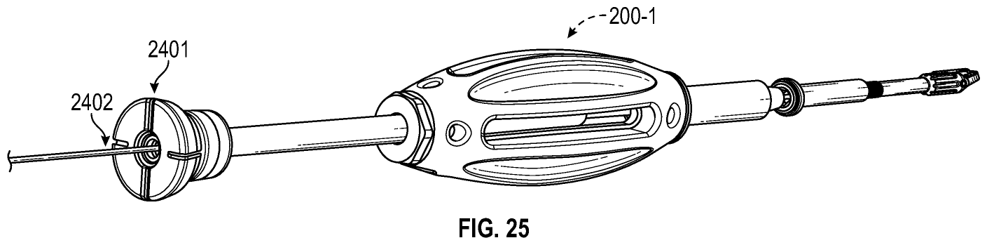

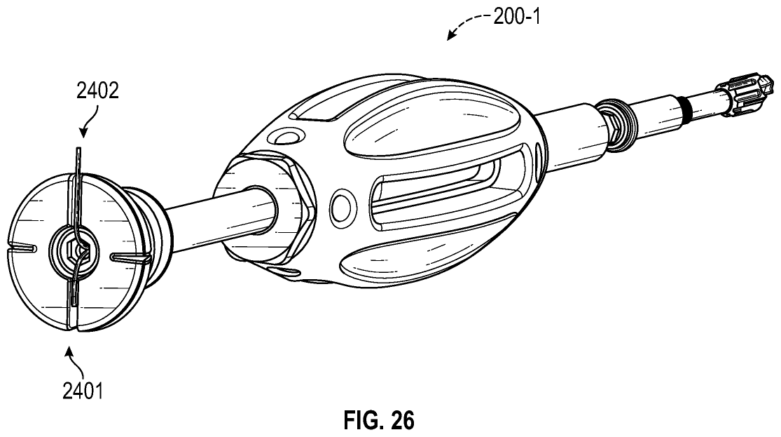

FIGS. 24 through 26 illustrate alternative perspective views of another system including an alternative inserter 200-1, according to an alternative embodiment of the present disclosure. The inserter 200-1 functions similar to inserter 200. For example, the inserter 200-1 may include one or more notches 2401 for actuating, securing, twisting, and/or tying an end of an actuator 2402. However, in some embodiments, the inserter 200-1 differs from the inserter 200 by using one or more different types of forces to engage, disengage, and/or insert an anchor member. For example, instead of using a compressive force to engage or disengage a component of a force-toggling tip, the inserter 200-1 may use a retractive or decompressive (e.g., pulling) force to engage or disengage a component of the force-toggling tip. Additionally, in some embodiments, the handle of system 200-1 may rotate freely (e.g., without requiring a compressive force at the head of the handle) such that notches 2401 may be used to engage the actuator 2402 during twisting and/or tying the ends of the actuator 2402 together, which may further secure a toggle mechanism in place. In other embodiments, the retractive or pulling force may be required to allow the handle to rotate freely to twist or tie the ends of the actuator.

Generally, the methods disclosed herein for implanting a system for providing compression between a first bone portion and a second bone portion include preparing a bone surface for surgery. This may include inserting a guide such as a K-wire through a first bone portion.

The methods may further include positioning a depth gauge relative to at least the first bone portion. The second elongated structure may provide a first depth measurement. In some embodiments, each of the K-wire and the depth gauge may be repeatedly used to provide additional levels of stability and/or additional depth measurements throughout the methods. In other embodiments, K-wire and/or the depth gauge may be used only once.

The methods may further include positioning a bone boring apparatus relative to the first bone portion. In some embodiments, the methods may include reinserting the depth gauge and/or the bone boring apparatus (e.g., having a different diameter drill bit), for additional depth measurements and/or bone boring.

The methods may include positioning a distal component of an anchor, such as the distal component 600 of the anchor 400, through the first bone portion and/or a the second bone portion.

The methods may optionally include positioning a proximal component of an anchor, such as the proximal component 700 of the anchor 400, relative to the first bone portion and/or the second bone portion. The distal end of the proximal component 700 may translatably couple to the distal component 600. The proximal end of the proximal component may include a proximal component. A diameter of the proximal component 700 may be larger than a diameter of the distal component 600. Accordingly, a diameter of the bored hole accommodating the distal component may be smaller than a diameter of the bored hole accommodating the proximal (e.g., female) component anchor member. Thus, the bone boring apparatus used to form the bone bore may be a stepped drill or other instrument designed to form a hole with a larger proximal diameter and a smaller distal diameter. The proximal end of the proximal component may include a drive feature (e.g., hexagonally shaped socket or drive head). The distal component 600 and the proximal component 700 may optionally be coupled together outside the bone and inserted as a unit, or inserted separately and assembled in situ.

The methods may further include positioning an inserter relative to the first bone portion and the proximal end of the proximal component. The inserter may be the inserter 200 described previously. The inserter may be used to insert and apply force (e.g., rotation, compression, and/or compaction) to the anchor 400. Application of the force to the anchor 400 may include a transfer of the force from the proximal component 700 to the distal component 600. The transfer of the force to the fourth elongated structure may include transfer of force (e.g., compressive force) to distal component. The transfer of compressive force to the distal component may include transferring force between bone surfaces of at least the first and second bone portions via the toggle mechanism 500 of the distal component 600 and the head 402 of the proximal component 700. As the inserter 200 may be secured to the proximal component 700, exertion of this compressive force may move the distal component 700 proximally, shortening the length of the anchor 400 to apply compression across the gap between the first bone portion 2701 and the second bone portion 2702.

The methods may further include actuating the toggle mechanism 500 using an actuator, such as the actuator 2002. The actuator 2002 may include a handle, a suture, a grip member, or combinations thereof. The actuation of the actuator 2002 may occur in a single intermediate step or in multiple intermediate steps (e.g., it may be repeated). The actuation of the actuator 2002 may provide additional stability and/or compressive force between the first bone portion and the second bone portion.

The methods may further include adjusting a length of the actuator 2002 to increase and/or decrease a distance between the toggle mechanism 500 and a surface of the second bone portion. The methods may further include the retraction and/or removal of the actuator.

In some embodiments, the methods may include adjusting a length of the anchor 400 by moving the proximal component 700 relative to the distal component 600. Additionally or alternatively, the methods may include adjusting a length of the anchor 400 by moving the toggle mechanism 500 of the distal component 600 relative to head 402 of the proximal component 700.

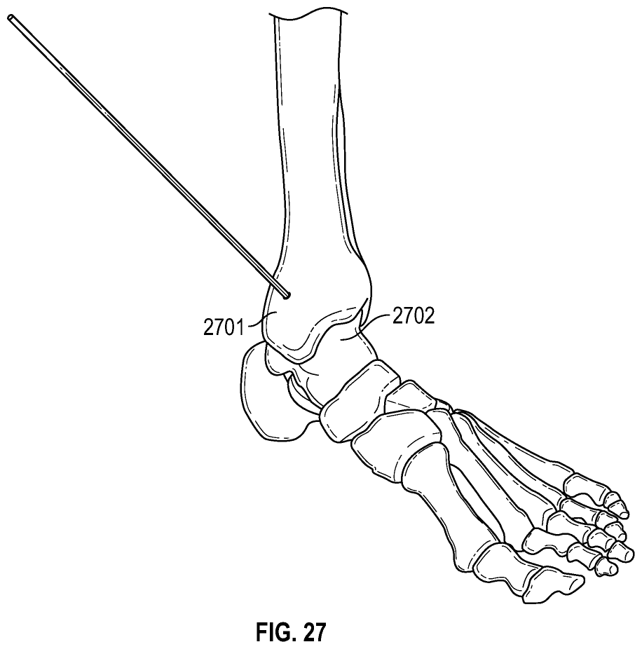

FIG. 27 is an antero-medial view of a partial tibia, talus, and general bone structure of a foot with a bone preparation tool positioned relative to the first bone portion 2701 (e.g., tibia) and the second bone portion 2702 (e.g., talus), according to an embodiment of the present disclosure. In some embodiments, a method of providing compressive force between a first bone portion and a second bone portion includes a preparatory step of positioning at least one of the first bone portion and the second bone portion in a desired orientation. For example, fractures may need to be repositioned to ensure optimum fixation and/or healing. Upon obtaining desired and/or optimum orientation of the first bone portion and the second bone portion, the K-wire may be positioned relative to the first bone portion 2701 and the second bone portion 2702. In some embodiments, this step of the method may include aspirating the joint using a needle, injection of pharmaceuticals (e.g., corticosteroid, anesthetics, etc.), use of a guide wire, guide arm, guide sleeve, and/or other intermediary and/or preparatory steps.

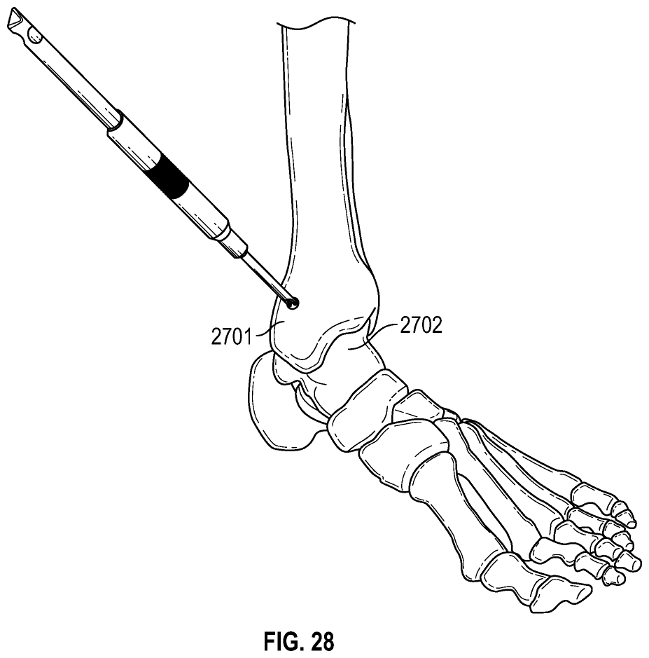

FIG. 28 is an antero-medial view of a partial tibia, talus, and general bone structure of a foot with a depth gauge positioned relative to the first bone portion 2701 and the second bone portion 2702. The depth gauge may include a laser range-finder and/or an analog depth gauge. In some embodiments of this step of the method, the depth gauge may be used relative to a bicortical hole through the first bone portion 2701 and the second bone portion 2702. In other embodiments, this step of the method may involve the use of one or more images (e.g., CT scan), or may be used without a hole through the bones.

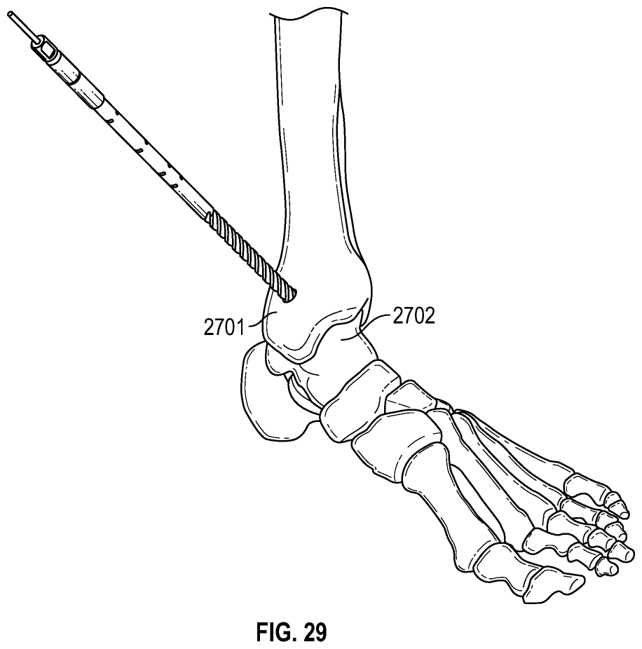

FIG. 29 is an antero-medial view of the partial tibia, talus, and general bone structure of the foot with a bone preparation tool (e.g., third elongated structure) positioned relative to the first bone portion 2701 and the second bone portion 2702. In some embodiments, the bone preparation tool includes a drill with a first attached drill bit, having a first diameter. In other embodiments, the bone preparation tool comprises a reamer.

In some embodiments, the first attached drill bit may be used to bore a hole in the first bone portion 2701 and the second bone portion 2702, where the hole has a diameter substantially equivalent to the first diameter along a desired trajectory. A second attached drill bit, having a second diameter may be used to bore a hole through at least the second bone portion 2702, but in some embodiments, through both the first bone portion 2701 and a portion of the second bone portion 2702. In some embodiments, the second diameter is smaller than the first diameter, (e.g. resulting underdrill of a far cortex and overdrill of a near cortex). In other embodiments, the second diameter is larger than the first diameter (e.g., resulting overdrill to at least a portion of both the near cortex and the far cortex). In yet other embodiments, more than two diameters of drill bits may be used to bore the hole through the first bone portion 2701 and the second bone portion 2702 along the desired trajectory.

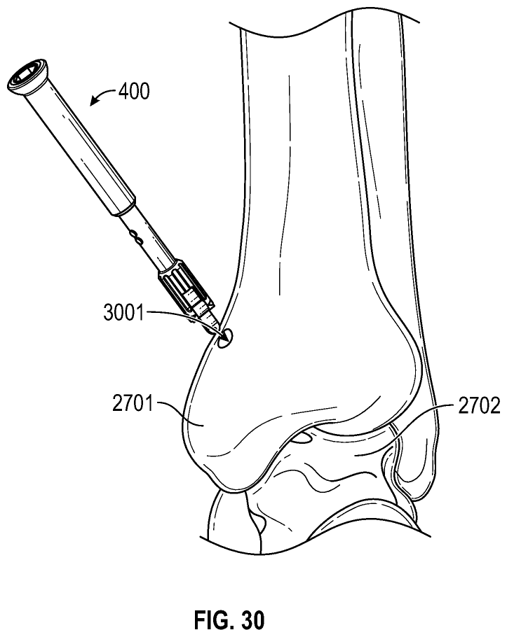

FIG. 30 is a view of the anchor 400 positioned for insertion into a tibiotalar joint through a hole extending obliquely through the first bone portion 2701 and the second bone portion 2702. The toggle mechanism of the anchor 400 is positioned in a retracted position and is configured to engage a portion of the second bone portion 2702 (e.g., bore surfaces beyond a diameter of the bore hole). In some embodiments, as illustrated in FIG. 30, the anchor 400 is configured and positioned to be inserted as a single elongated structure. In other embodiments, the distal, or male, component is inserted independent of the proximal, or female, component. For example, the distal component may be inserted into hole 3001 detached from the proximal component of the anchor 400. In other embodiments, the distal component is inserted into hole 3001 simultaneously with the proximal component (e.g., with the proximal component attached to the distal component). A depth gauge, hole geometry, and/or image (e.g., CT scan) may help determine when the anchor 400, or a component thereof, has reached the optimum depth of insertion.

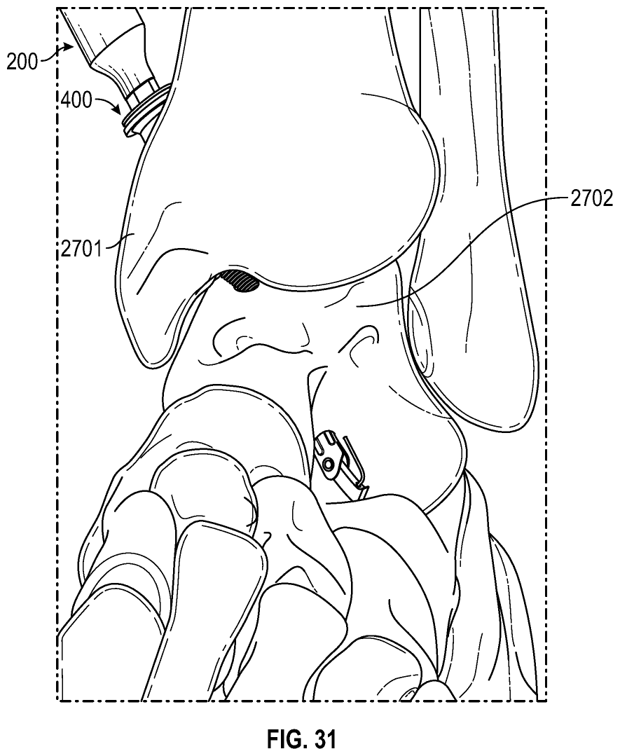

FIG. 31 is a close-up view of the anchor 400 inserted into the tibiotalar joint through a hole extending obliquely through the first bone portion 2701 and the second bone portion 2702. In this step of the method, the toggle mechanism may be positioned to engage another portion of the second bone portion 2702 (e.g., cortex or bone surface of the talus). In some embodiments, the inserter 200 of the system 100 may be used to help the anchor 400 obtain the optimum depth within the hole 3001. In other embodiments, the inserter 200-1, anchor 400-1, and/or anchor 400-2 may be used in the system 100.

In some embodiments, this step of the method includes advancing the proximal component 700 with respect to the distal component 600 and toggle mechanism 500 by advancing the handle of the inserter (turning clockwise in this case), while holding and keeping handle intact (e.g., applying compressive force). As, the distal component 600 advances toward the proximal component 700, the construct may generate a force to compress the proximal and distal bone portions (e.g., FIGS. 52 through 54).

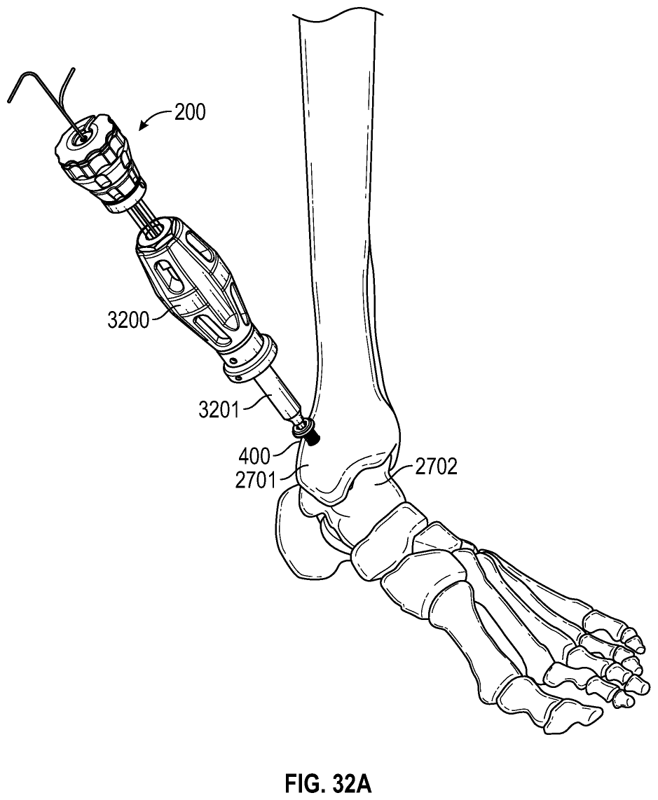

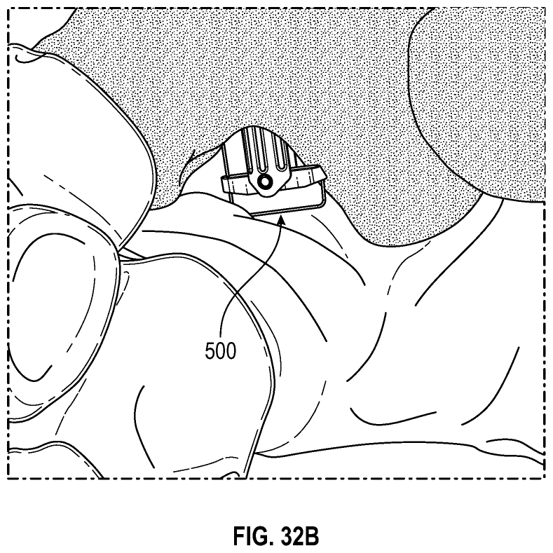

FIG. 32A is an antero-medial view of a partial tibia, talus, and general bone structure of a foot with the system of FIG. 23 positioned to engage the anchor 400. In this step of the method, the proximal component 700 of the anchor 400 exhibits prominence. FIG. 32B illustrates a close-up view of a distal component (e.g., toggle mechanism) positioned for engaging a portion of the second bone portion 2702. In some embodiments, the prominence of the proximal component is likely due or related to a prominence of the distal component, or the toggle mechanism 500, as depicted in FIG. 32B. This step of the method may include the detection or determination of prominence of at least one of the distal component and the proximal component. A grip member 3200 of the inserter 200 may be used to transfer force to a drive shaft 3201. The application of force at this step in the method may be used to reduce and/or eliminate the detected/determined prominence.

In some embodiments, the grip member 3200 may be similar and/or identical to first grip member 1100. In other embodiments, the grip member 3200 differs from the first grip member 1100 by the addition and/or subtraction of at least one component. For example, the grip member 3200 may be larger/smaller than the first grip member 1100 and may not include the biasing member 1000 of the first grip member 1100. In some embodiments, the drive shaft 3201 may be similar and/or identical to drive shaft 301. In other embodiments, the drive shaft 3201 differs from the drive shaft 301 by the addition and/or subtraction of at least one component. For example, the drive shaft 3201 may be larger/smaller and may not include the aperture 302 of the drive shaft 301.

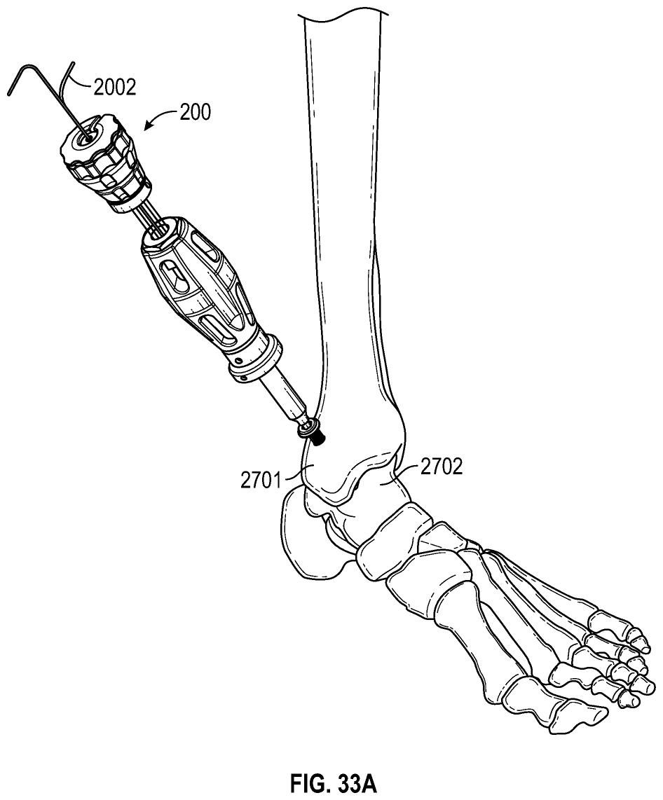

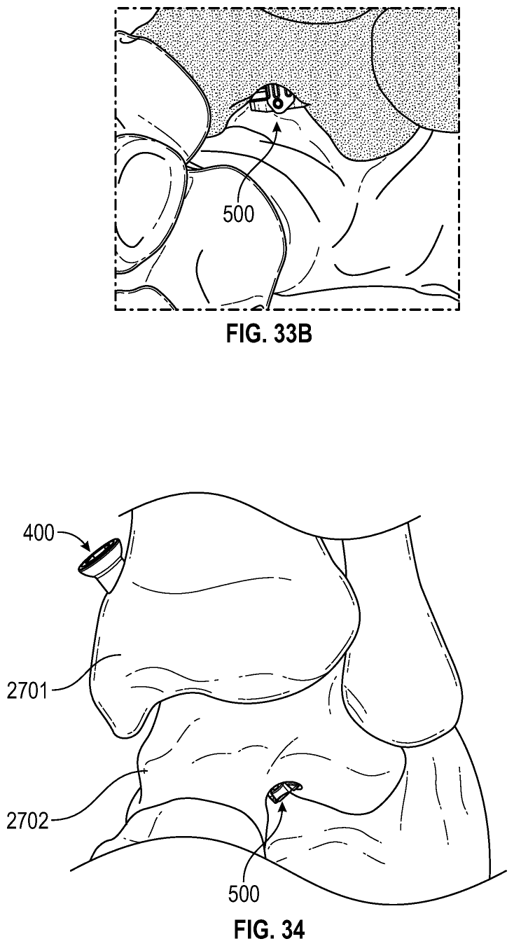

FIG. 33A illustrates another step in the method, or an intermediate step of the method step discussed with respect to FIG. 32A. For example, an actuator 2002 of the inserter 200 of system 100 may be positioned before and/or during the rotation of the drive shaft 3201 to transfer force to the anchor 400. The actuator 2002 may be actuated (e.g., pulled) to move the toggle mechanism into a deployed position. In some embodiments, one or more ends of the actuator 2002 are fed out of a proximal end (e.g., handle head) of the inserter 200 during transfer of the first rotational force to the anchor 400. For example, while the grip member 3200 is rotated, the anchor 400 may be reducing in length, such that the actuator 2002 may be simultaneously fed out of the proximal end of the inserter 200. FIG. 33B may illustrate a close-up view of the toggle mechanism 500. The toggle mechanism 500 is positioned for engaging a portion of the second bone portion 2702, or it is in a deployed position. The prominence of the toggle mechanism 500 may be reduced during this step in the method, such that the elongated member moves closer in proximity to the distal cortex (e.g., bone surface of the second bone portion 2702).

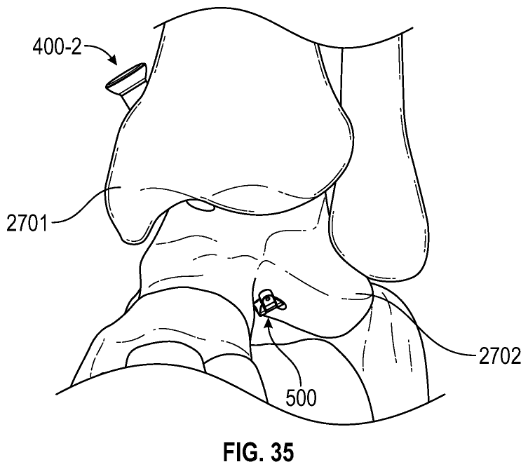

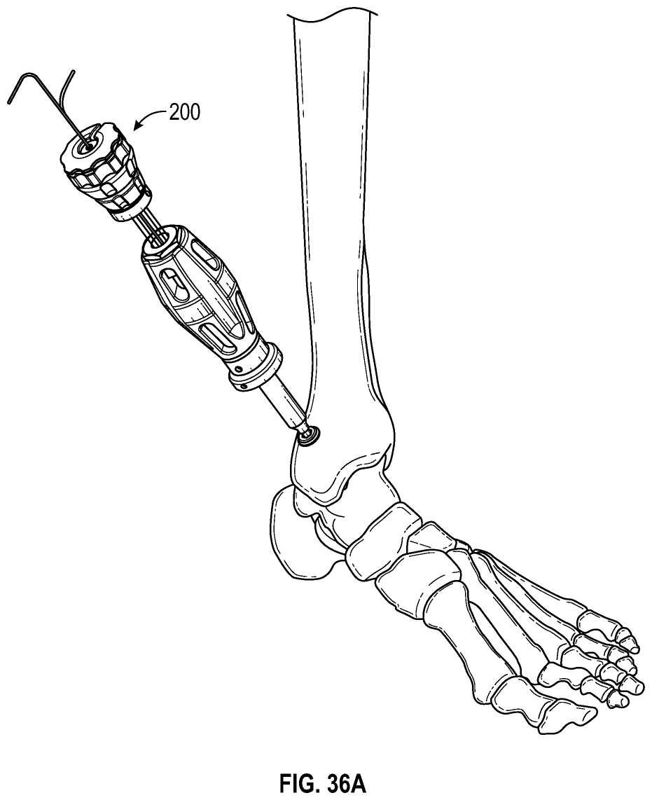

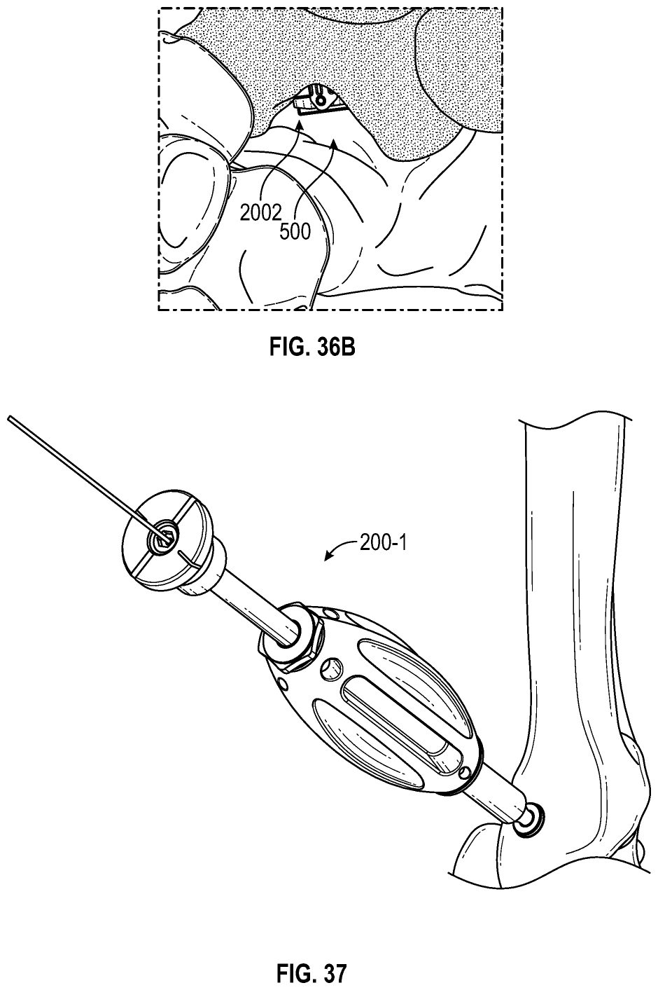

FIGS. 34 and 35 are different views of alternative embodiments of the anchor member (e.g., anchor 400 and 400-2) extending obliquely through the first bone portion 2701 and the second bone portion 2702, where the toggle mechanism 500 is engaging a bone surface of the second bone portion 2702. Each of the proximal components of the anchor members are exhibiting prominence. In some embodiments, the inserter 200 (e.g., inserter) may be used to apply additional rotational force, as depicted in FIG. 36A, to reduce and/or eliminate this prominence. After the prominence of the anchor member has been reduced, the ends of the actuator 2002 may be tied off, providing additional stability to the anchor member and additional compressive force between the first bone portion 2701 and the second bone portion 2702. As illustrated in FIG. 36B, the actuator 2002 is securely seated against the elongated member of the toggle mechanism 500 after the actuator 2002 has been tied off. Significantly, the path of the actuator 2002 through holes in the toggle mechanism allows for a subsequent retraction and/or removal of the actuator 2002. This provides a surgeon with an added degree of customization (e.g., leaving or removing the actuator) of the system 100 based on the needs of a patient and/or the situation of the first bone portion 2701 and/or the second bone portion 2702.

In other embodiments, an alternative inserter 200-1 may be used to reduce the prominence exhibited by an anchor member (as depicted in FIGS. 34 and 35). For example, FIG. 37 is an antero-medial view of a partial tibia and talus with the system of FIG. 24 positioned to engage and rotate an anchor, thereby reducing prominence of the anchor, according to another embodiment of the present disclosure.

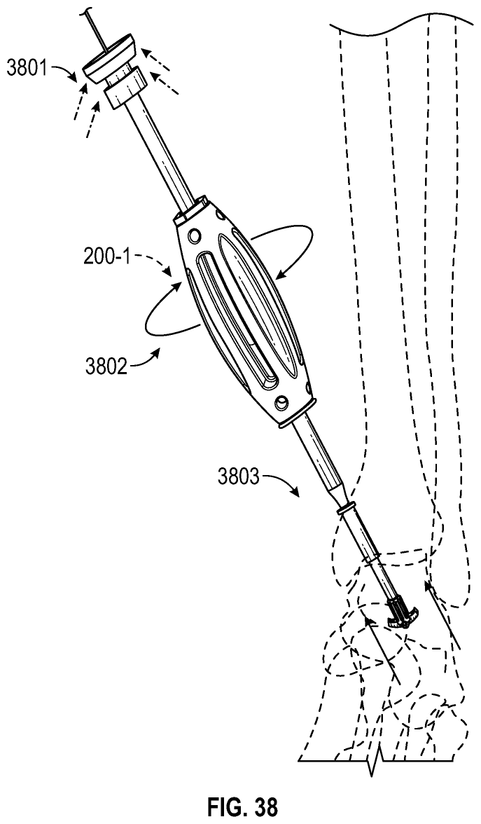

FIG. 38 illustrates an intermediate step of the method step depicted and described with respect to FIG. 37. For example, FIG. 38 is an antero-medial view of a partial tibia and talus with the system of FIG. 24 positioned to engage and rotate the anchor, having the anchor positioned obliquely through a portion of the tibiotalar joint. A retraction force 3801 may be applied to a handle of the inserter 200-1. In some embodiments, due to the manner in which the actuator may be positioned relative to the handle, this retraction force 3801 may move the toggle mechanism 500 into a deployed position. In other embodiments, the retraction force 3801 may enable the first grip member to receive a rotational force.

Upon moving the toggle mechanism 500 into its deployed position, a rotational force 3802 may be applied to the grip member of the inserter 200-1. Upon receipt of the rotational force 3802, rotational force (not shown) may be applied to the drive shaft and anchor, which thereby draws the toggle mechanism (and second bone portion 2702) towards the proximal component (e.g., head) of the anchor member as indicated by the arrows 3803. In other words, the distal (male) component of the anchor member advances into the proximal member (e.g., via correlating coupling features). In some embodiments, this direction of advancing compressive force results in the distal bone fragment being compressed into the proximal bone fragment. For example, a displaced intra-articular fracture may be engaged and moved by the distal, or male, component of the anchor, and moved or compressed proximally, or towards the more stable bone, such as a femur or proximal tibia.

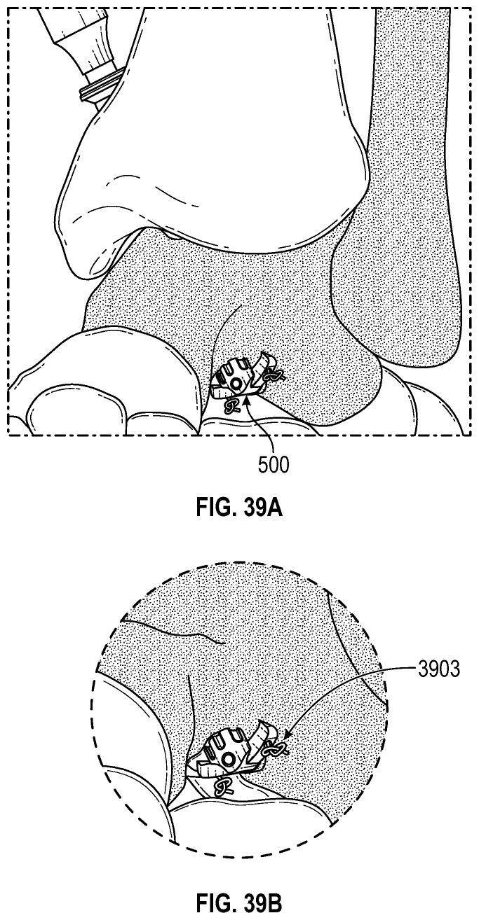

In some embodiments, the methods described above may be preceded by securing one or more ends of the actuator 2002. For example, FIGS. 39A and 39B illustrate a close-up views of an anchor and the toggle mechanism 500 engaging the tibia and talus, according to an embodiment of the present disclosure. The ends 3903 of the sutures used as the actuator for actuating the toggle mechanism 500 are tied. Although tying off the ends 3903 may reduce or eliminate the possibility of removing the actuator, this added step may add additional stability relative to inserting the anchor and toggle mechanism and/or provide additional compressive or stabilizing force relative to the first bone portion and/or the second bone portion. In other embodiments, the step of securing the one or more ends of the actuator follows the steps of the methods previously discussed (e.g., follows the proximal movement of the distal male component towards the proximal female component).

In the alternative to tying off the ends 3903 to the toggle mechanism 500, the ends 3903 may be tied to other locations on the anchor 400. For example, the ends 3903 may be tied to the proximal component 700, or more precisely, to the head 402. If desired, the head 402 may have one or more slits, clamps, and/or other features (not shown) designed to facilitate retention of the ends 3903 on the head 402.

FIG. 40 is an antero-medial view of a partial tibia, talus, and general bone structure of the foot, with a distal component of a distal end of the anchor engaging the tibia, according to an embodiment of the present disclosure. At this step in the method, the inserter 200 has been removed from the anchor 400, with the actuator 2002 remaining in place. This is possible due to the cannulated structure of each component of the proximal member, thereby allowing the proximal member to slide off of the actuator 2002 after the anchor has been fully inserted into the hole in the bones. The length of the actuator provides sufficient length for the surgeon to tie, stitch, twist, or further manipulate the actuator 2002 as desired. In some embodiments, this step in the method includes unwinding the suture strands from the inserter instrument and tugging proximally to flip the toggle mechanism with respect to the distal component of the anchor. In other embodiments, this step in the method includes unwinding the suture strands and tugging proximally to remove the suture strands.

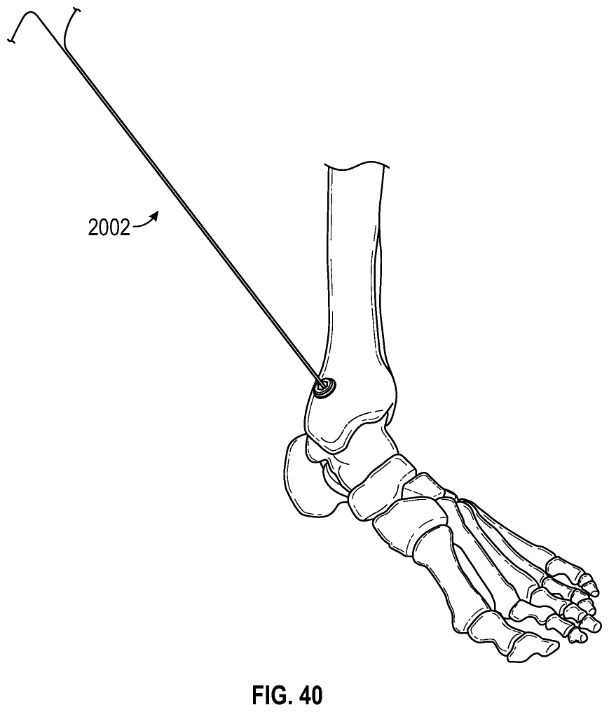

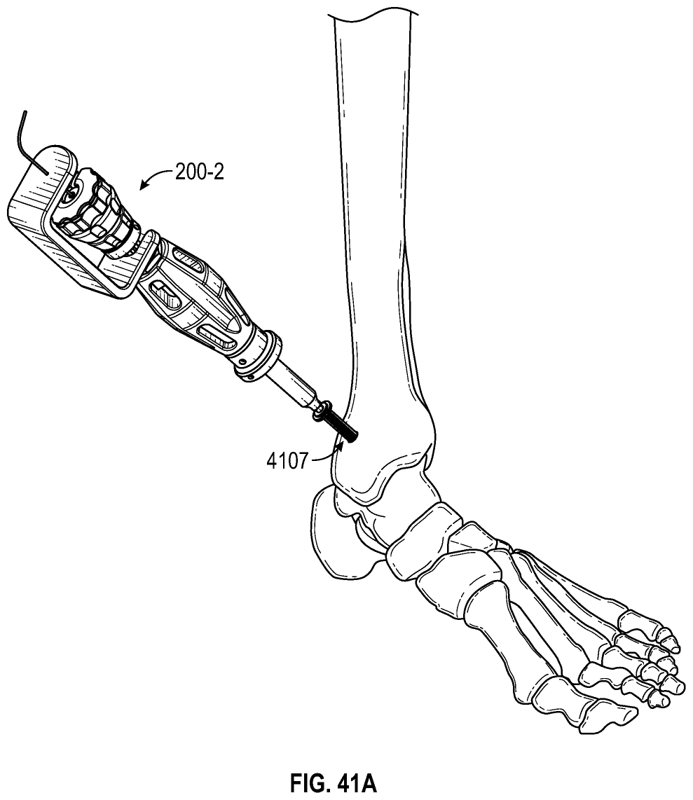

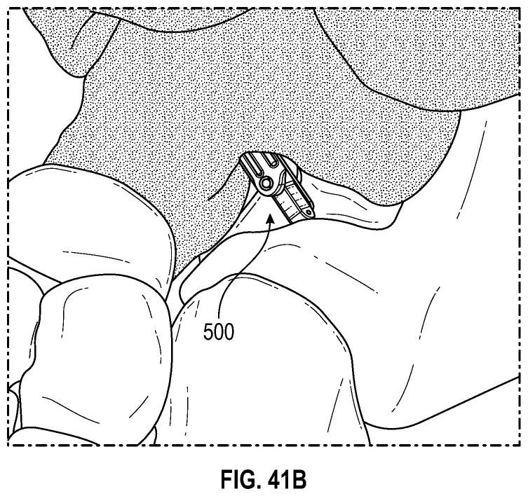

In other embodiments, a prominence of an anchor may be detected and may require additional force to be reduced and/or eliminated. For example, FIG. 41A illustrates an antero-medial view of a partial tibia, talus, and general bone structure of a foot with another system positioned to engage an anchor. The anchor exhibits extreme prominence by having a standoff distance 4107 relative to the tibia. As depicted in FIG. 41B, the prominence of the proximal component, or head of anchor 400, may position the distal component, or toggle mechanism 500, relative to an adjacent bone surface (e.g., navicular bone) in a manner that prevents moving the toggle mechanism 500 into a deployed position. Therefore, in some embodiments of the system for providing compressive force between a first bone portion and a second bone portion, a compaction receiving member is included and attached to the proximal member of the system.

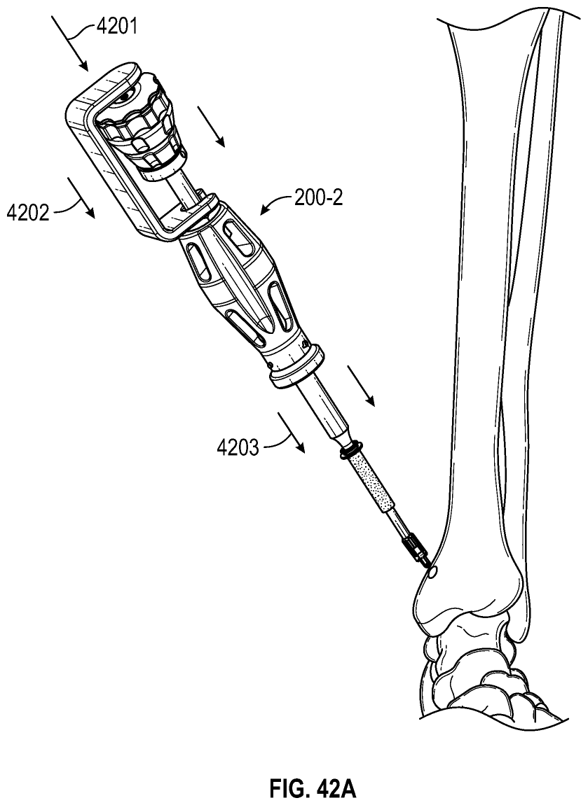

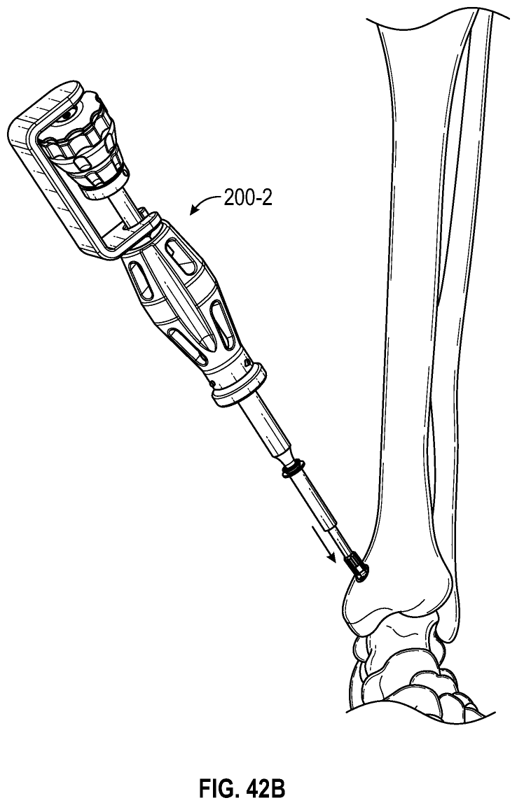

For example, referring now to FIG. 42A, a inserter 200-2 may include a compaction or impact plate attached to the inserter 200-2. The compaction or impact plate is made of a hardened metal to resist subsequent bending after its formation. For example, the impact plate may comprise stainless steel and/or titanium. A distal end of the impact plate may abut a hard, flat surface of the inserter 200-2, such as flat surface 205 of the threaded component 800 of inserter 200. The distal end of the impact plate may include an opening for being removably connected to the inserter 200-2.

In some embodiments, the proximal end of the impact plate is positioned in proximity, without abutting, the handle of the proximal member. In other embodiments, the proximal end of the impact plate abuts the proximal end, or knob, of the handle.

The proximal end of the impact plate may include a flat surface area that is configured to receive an impact force 4201. This impact force 4201 may be transferred as an impact force 4202, parallel to the impact force 4201, to a threaded component such as the threaded component 800 of the inserter 200. The attachment of the threaded component to the female end of the drive shaft (e.g., similar, or identical to drive shaft 301) allows the for the impact force 4202 to be transferred along the drive shaft as another impact force 4203 parallel to the anchor member. In some embodiments, these forces, 4201, 4202, and 4203 allow an anchor member, such as the anchor depicted in FIGS. 42A and 42B, to receive an impact force through a portion of, or the entire, insertion process of the anchor member. In other embodiments, these impact forces 4201, 4202, and 4203 may be useful in moving a toggle mechanism, such as toggle mechanism 500 in FIG. 41B, that has been prevented from actuation into its deployed position.

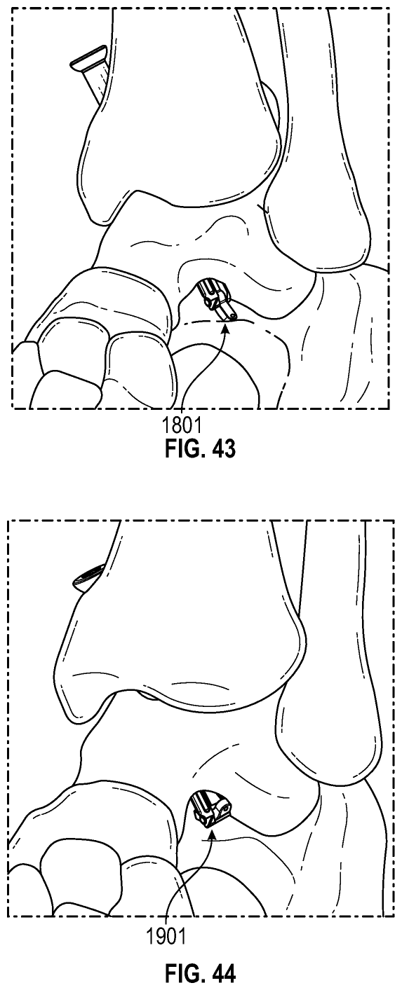

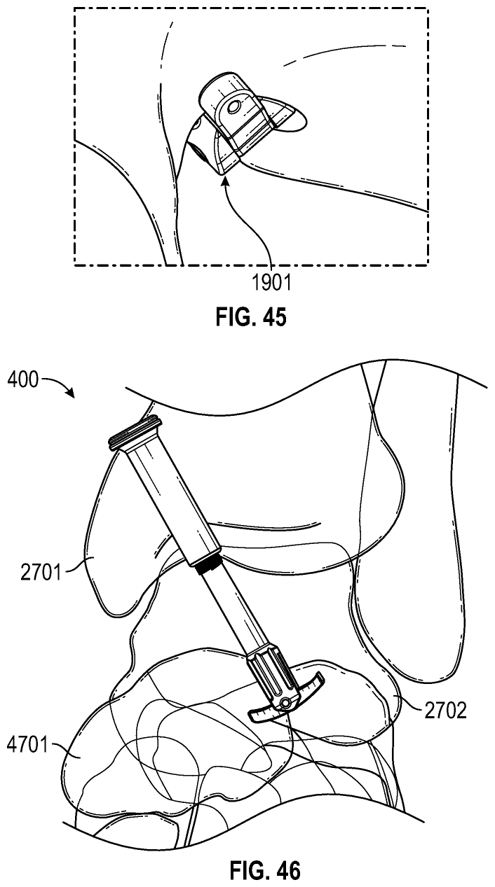

FIG. 43 is a close-up view of an anchor in a retracted position relative to the tibia and talus, that is in a retracted position 1801. The anchor in FIG. 43 is being prevented from movement into a deployed position due to the proximity of an adjacent bone. FIG. 44 is a close-up view of an anchor in the engaged position 1901 upon receiving at least one of the impact forces 4201, 4202, and 4203. As depicted in FIG. 44, in some embodiments, the distal component may exhibit prominence after receiving the at least one of the impact forces 4201, 4202, and 4203. As previously discussed, the proximal component may include a head with a drive feature for further rotating the proximal component to draw the distal component (e.g., toggle mechanism) towards the proximal component. Upon receipt of the additional rotational forces, the prominence of the distal component may be reduced and/or eliminated, as depicted in FIG. 45, where the elongated toggle mechanism directly abuts a bone surface of the second bone portion 2702.

Significantly, the impact forces 4201, 4202, and 4203 may be provided in situations where the toggle mechanism is not being prevented from being moved into a deployed position. For example, the proximal component 700 may be experiencing friction, which may be overcome by the application of the impact forces 4201, 4202, and 4203.

Upon receipt of additional rotational forces, an image such as the CT scan depicted in FIG. 46, may be used to depict the anchor 400 relative to the first bone portion 2701, the second bone portion 2702, and/or an additional bone, such as the calcaneus. This step may help a surgeon determine if additional rotational and/or impact forces may be necessary to complete the insertion of the anchor implant.

In other embodiments, the methods disclosed herein may generally include assembling an anchor member, translatably coupling a proximal component to a distal anchor member, positioning an actuator relative to a distal end and a proximal end of the anchor member, inserting the anchor member as independent or attached components relative to a first bone portion and a second bone portion, and actuating a toggle mechanism of the anchor member into a deployed position. The methods may optionally include determining a position of the deployed toggle mechanism relative to at least the second bone portion. The methods may further include applying a first rotational force relative to a distal end of the anchor member. The methods may further include applying additional rotational force, or continuing to apply the first rotational force, until a dynamic compression ability of the washer and/or the elongated member of the toggle mechanism is obtained.

The following steps illustrate a method for bone fixation using the device described above and the related instrumentation.

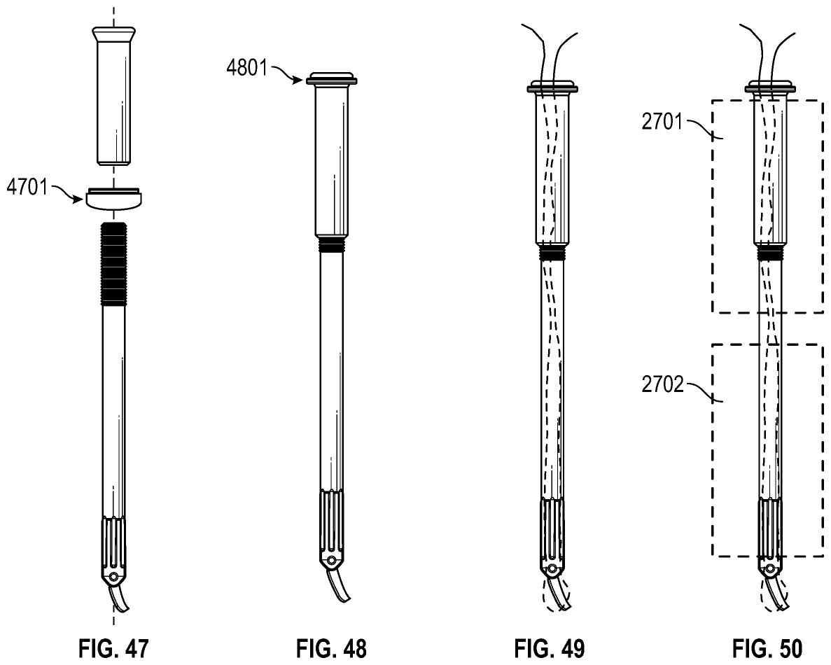

Referring now to FIG. 47, in a first step of the additional method embodiments, an anchor member is assembled. For example, an anchor 400-1, may be assembled to include a curved compression washer and/or an elastic/superelastic elongated member. The curved compression washer may be in an unbiased state 4701.

Referring now to FIG. 48, in a second step of the additional method embodiments, a proximal component is translatably coupled to a distal anchor member. In this step, or a subsequent step, the compression washer may assume a biased state 4801.

Referring now to FIG. 49 in a third step, an actuator is positioned relative to a distal end and a proximal end of the anchor member. In some embodiments, this third step precedes the second step. In other embodiments, this third step follows the second step. In some embodiments, this third step may include threading a suture into holes of the toggle mechanism based on a desired direction of rotation of the toggle mechanism. For example, when the toggle mechanism is desired to rotate in a dorsal direction, the suture may be threaded in a first direction through a proximal hole, enter a distal hole, then exit the proximal hole in a second direction. When the toggle mechanism is desired to rotate in a plantar direction, the threading pattern may be reversed in at least one aspect (e.g., exit the distal hole in the first direction).

Referring now to FIG. 50, in a fourth step, the additional method embodiments may include inserting the anchor member as independent component. For example, a distal or male anchor component may be inserted detached from the proximal female component. In other embodiments, the distal male anchor component may be inserted after it has been translatably coupled to the proximal female anchor component. The insertion of the anchor components, whether as attached or independent members, occurs relative to the first bone portion 2701 and the second bone portion 2702, with the proximal component adjacent the first bone portion and the distal component adjacent the second bone portion. In some embodiments, the insertion of the anchor component is an oblique insertion along a single trajectory through the tibia and the talus. In other embodiments, the insertion may be a lateral, medial, medial-lateral, anterior, or other type of insertion. In some embodiments, this fourth step includes compressing the compression washer against the first bone portion 2701, such that the washer assumes the biased state 4801.

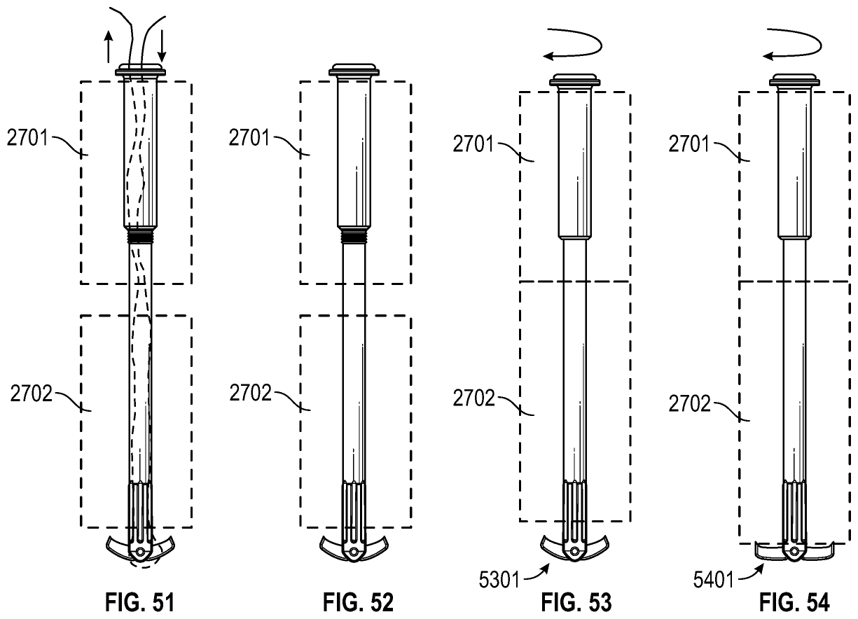

Referring now to FIG. 51, in a fifth step a toggle mechanism of the anchor member is actuated and/or moved from a retracted position into a deployed position. This fifth step may include pulling on one or both ends of the actuator and rotating an elongated member of the toggle mechanism until it is substantially parallel with a surface of the second bone portion.

Referring now to FIG. 52, the additional method embodiments may optionally include a sixth step. The optional sixth step may include determining a position of the deployed toggle mechanism relative to at least the second bone portion 2702. This optional sixth step may include obtaining one or more images, such as an X-ray image, a CT scan, or combinations thereof. The optional sixth step may occur to ensure that the elongated member of the toggle mechanism and the components of the anchor member or in optimum positions relative to the first bone portion and the second bone portion, requiring no further adjustment. The optional sixth step may further include repositioning at least one of the first bone portion 2701, the second bone portion 2702, the proximal component, the distal component, and/or the toggle mechanism.

Referring now to FIG. 53, the additional method embodiments may further include a seventh step. The seventh step may include applying a first rotational force first relative to a distal end of the anchor member. In some embodiments, the first rotational force may cause the proximal component to abut the first bone portion 2701. In other embodiments, the first rotational force may cause the distal component to abut the second bone portion 2702. In yet other embodiments, the first rotational force causes a length between the proximal component and distal component to decrease without (at least initially) abutting the first bone portion 2701 or the second bone portion 2702. In some embodiments, in the seventh step, the elongated member assumes an unbiased state 5301.

Referring now to FIG. 54, the additional method embodiments may further include an eighth step. The eighth step may include applying an additional force, or continuing to apply the first rotational force, after initially receiving the first rotational force. In some embodiments, the additional force applied to the anchor component may include an impact force. In other embodiments the additional force applied may be a continuation of the application of the first rotational force or a second rotational force. These additional forces are applied, or the first rotational force is continued after its initial application, until a dynamic compression ability of the washer and/or the elongated member of the toggle mechanism is obtained. For example, one or both of the curved washer and the elongated member may flatten, reducing or eliminating the manufactured curve, thereby providing a dynamic compressibility to the anchor member. In some embodiments, in this eighth step, the elongated member of the toggle mechanism assumes a biased state 5401.

For example, the elongated member of the toggle mechanism may comprise a flexible material/super elastic material. After the gap between the first bone portion 2701 and the second bone portion 2702) is closed and compression is achieved, further advancement of the distal component 600 toward the proximal component 700 will result in the elongated member of the toggle mechanism 500 flexing from an arcuate or crescent shape (e.g., unbiased state 5301) to a straight or linear shape (e.g., biased state 5401) as shown in FIG. 54. The energy generated in flexing the toggle mechanism from arcuate to crescent shape may generate a continuous (e.g., dynamic) compression relative to the bone fragments and/or joint.

In some embodiments, an arthroplasty system may be converted to an arthrodesis system if desired. For example, a spacer shaped to occupy the footprint and height of a tibial plate, bearing insert, and talar plate may be inserted to replace those components, and a single compression bolt, passing through the spacer window, may connect to the talar anchor to provide compression across the joint and provide fusion. Additional method steps for converting from ankle arthroplasty to arthrodesis are described in U.S. Pat. No. 9,962,201, entitled “JOINT ARTHRODESIUS AND ARTHROPLASTY,” which issued on May 8, 2018, which is incorporated by reference herein in its entirety.

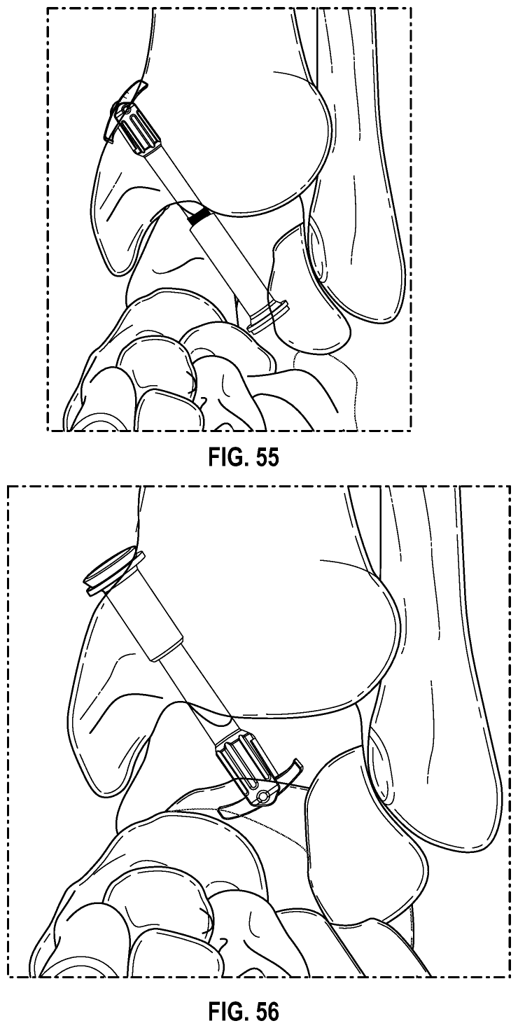

Referring to FIG. 55, an anterior view depicts the use of an anchor like the anchor 400 described above, in a retrograde approach. The systems and methods of the present disclosure are not limited to use in any particular procedure or surgical approach. Some surgical procedures may be reasonably doable from either of two opposite directions. For example, in some embodiments, the ankle arthrodesis mentioned above may be carried out by inserting the anchor 400 along a distal/lateral approach, rather than with the proximal/medial approach set forth above. The steps for inserting the anchor 400 with this approach may generally be as set forth above, except that the tibia may be pulled distally and laterally toward the talus, rather than the opposite, as set forth above.

Referring to FIG. 56, another anterior view illustrates the use of the anchor 400 to secure bones together without passing out of the second bone portion. Rather than inserting the anchor 400 into a hole passing completely through the first bone portion and the second bone portion, the surgeon may instead form a blind hole terminating in the interior of the second bone portion. Upon insertion of the anchor 400, the distal component 700 may terminate in the interior (for example, in the cancellous bone) of the second bone portion, and the toggle mechanism 500 may deploy within the cancellous bone. Then, the toggle mechanism 500 may lodge against the cancellous bone to provide the desired compression between the first and second bone portions. If desired, the length of the anchor 400 may be selected such that the toggle mechanism 500 lodges against the interior cortex of cortical bone within the second bone portion. Advantageously, this method may leave the exterior of the second bone portion (in this example, the talus) undisturbed, and may require no surgical access to this location.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

Reference throughout this specification to “an embodiment” or “the embodiment” means that a particular feature, structure or characteristic described in connection with that embodiment is included in at least one embodiment. Thus, the quoted phrases, or variations thereof, as recited throughout this specification are not necessarily all referring to the same embodiment.

Similarly, it should be appreciated that in the above description of embodiments, various features are sometimes grouped together in a single embodiment, Figure, or description thereof for the purpose of streamlining the disclosure. This method of disclosure, however, is not to be interpreted as reflecting an intention that any claim requires more features than those expressly recited in that claim. Rather, as the following claims reflect, inventive aspects lie in a combination of fewer than all features of any single foregoing disclosed embodiment. Thus, the claims following this Detailed Description are hereby expressly incorporated into this Detailed Description, with each claim standing on its own as a separate embodiment. This disclosure includes all permutations of the independent claims with their dependent claims.

Recitation in the claims of the term “first” with respect to a feature or element does not necessarily imply the existence of a second or additional such feature or element. Elements recited in means-plus-function format are intended to be construed in accordance with 35 U.S.C. § 112 Para. 6. It will be apparent to those having skill in the art that changes may be made to the details of the above-described embodiments without departing from the underlying principles set forth herein.

While specific embodiments and applications of the present disclosure have been illustrated and described, it is to be understood that the scope of the appended claims is not limited to the precise configuration and components disclosed herein. Various modifications, changes, and variations which will be apparent to those skilled in the art may be made in the arrangement, operation, and details of the methods and systems disclosed herein.

1. A system for providing compressive force relative to a first bone portion and a second bone portion, the system comprising:
an anchor insertable into a hole formed through at least part of the first bone portion and the second bone portion, the anchor comprising:
a proximal component comprising:
a proximal component proximal end comprising a head; and
a proximal component distal end comprising a proximal coupling feature;
a distal component comprising:
a distal component proximal end comprising a distal coupling feature that is configured to be translatably coupled to the proximal coupling feature;
a distal component distal end comprising a toggle mechanism that is movable between:
a retracted position, in which the toggle mechanism is insertable through the hole; and
a deployed position, in which the toggle mechanism extends beyond a diameter of the hole to restrict motion of the toggle mechanism through the hole; and
an actuator configured to be operatively coupled to the toggle mechanism such that the actuator can be actuated by a user to trigger motion of the toggle mechanism between the retracted position and the deployed position.

2. The system of claim 1, further comprising an inserter comprising a grip member and a biasing member configured to compress to transmit rotation of the grip member to the anchor.

3. The system of claim 1, wherein the toggle mechanism comprises a curved elongated member.

4. The system of claim 3, wherein the curved elongated member is formed of a superelastic material.

5. The system of claim 1, wherein:
the toggle mechanism comprises an elongated member; and
the actuator comprises a suture coupled to the elongated member such that tension on the suture causes the elongated member to rotate from the retracted position to the deployed position.

6. The system of claim 1, wherein the proximal coupling feature comprises internal threads, and the distal coupling feature comprises a male member having proximal external threads configured to translatably engage the internal threads of the proximal coupling feature.

7. A system for providing compressive force relative to a first bone portion and a second bone portion, the system comprising:
an anchor insertable into a hole formed through at least part of the first bone portion and the second bone portion, the anchor comprising:
a proximal component comprising:
a proximal component proximal end comprising a head; and
a proximal component distal end comprising a proximal coupling feature;
a distal component comprising:
a distal component proximal end comprising a distal coupling feature that is configured to be translatably coupled to the proximal coupling feature;
a distal component distal end comprising a toggle mechanism that is actuatable to move between:
a retracted position, in which the toggle mechanism is insertable through the hole; and
a deployed position, in which the toggle mechanism extends beyond a diameter of the hole to restrict motion of the toggle mechanism through the hole;
wherein the anchor is configured to elastically deform in response to exertion of the compressive force such that the anchor continues to exert at least part of the compressive force if the first bone portion and the second bone portion move closer together after implantation of the anchor.

8. The system of claim 7, wherein the toggle mechanism comprises an elongated member that is pivotably coupled to a remainder of the distal component such that the elongated member is pivotable from the retracted position to the deployed position.

9. The system of claim 8, wherein the elongated member is configured to elastically deform in response to exertion of the compressive force.

10. The system of claim 9, wherein the elongated member comprises:
a central portion that is pivotably coupled to the remainder of the distal component; and
two end portions that, in the deployed position, contact the second bone portion;
wherein the elongated member is configured to elastically deform such that the end portions flex distally relative to the central portion.

11. The system of claim 10, wherein each of the end portions comprises a spike that, in the deployed position, is oriented toward the second bone portion.

12. The system of claim 11, wherein the elongated member is formed of a superelastic material.

13. The system of claim 12, wherein:
the proximal component distal end comprises an internally-threaded bore; and
the distal component distal end comprises a threaded post configured to threadably engage the internally-threaded bore.

14. A system for providing compressive force relative to a first bone portion and a second bone portion, the system comprising:
an anchor moveable through a hole formed in at least part of the first bone portion or the second bone portion, the anchor comprising:
a proximal component comprising:
a proximal component proximal end comprising a head; and
a proximal component distal end comprising a bore;
a distal component comprising:
a distal component proximal end comprising a shaft that is configured to be translatably retained within the bore;
a distal component distal end comprising a toggle mechanism that is moveable between:
a retracted position, in which the toggle mechanism is insertable through the hole; and
a deployed position, in which the toggle mechanism extends beyond a diameter of the hole to restrict motion of the toggle mechanism through the hole.

15. The system of claim 14, wherein the toggle mechanism is elastically deformable to help maintain compression between the first bone portion and the second bone portion after implantation.

16. The system of claim 15, further comprising a curved washer configured to encircle the proximal component and abut the first bone portion, wherein the curved washer is elastically deformable to further help maintain compression between the first bone portion and the second bone portion.

17. The system of claim 16, wherein the curved washer comprises a Nitinol washer.

18. The system of claim 14, wherein the proximal component comprises a maximum width, excluding the head, that is greater than a maximum width of the distal component such that the distal component fits within a distal portion of the hole with a distal diameter smaller than a proximal diameter of a proximal portion of the hole.

19. The system of claim 18, further comprises an inserter configured to be coupled to the proximal component and to, with the anchor in the hole, actuate the distal component proximally to draw the second bone portion toward the first bone portion.

20. The system of claim 19, further comprising an actuator configured to be operatively coupled to the toggle mechanism such that the actuator can be actuated by a user to trigger motion of the toggle mechanism between the retracted position and the deployed position.
